(19) United States
(12) Patent Application Publication
(10) Pub. No.: US 2018/0298344 A1
(43) Pub. Date: Oct. 18, 2018

(54) Realistic Humanized Model of Disease Matrix Environment for Evaluation of Therapeutic Reversal or Cell Fate

(71) Applicant: Yale University, New Haven, CT (US)

(72) Inventors: Parid Sava, New Haven, CT (US); Anjelica Gonzalez, New Haven, CT (US)

(21) Appl. No.: 15/950,354

(22) Filed: Apr. 11, 2018

Related U.S. Application Data
(60) Provisional application No. 62/484,455, filed on Apr. 12, 2017.

(51) Int. Cl. C12N 5/071; C12N 5/077

(52) U.S. Cl. CPC C12N 5/0691; C12N 5/0656; C12N 2503/02; C12N 2506/28; C12N 2533/90; C12N 2533/30; C12N 2513/00

(57) ABSTRACT

The present invention relates to tunable hydrogels generated by selecting an amount of polymer and cross-linker such that the resulting hydrogel when covalently linked to an extracellular matrix (ECM) provides a biomimetic three-dimensional environment.

### CROSS REFERENCE TO RELATED APPLICATIONS

This application claims priority to U.S. Provisional Application No. 62/484,455, filed Apr. 12, 2017 which is hereby incorporated by reference herein in its entirety.

### BACKGROUND OF THE INVENTION

Idiopathic pulmonary fibrosis (IPF) is a lung disease with no curative treatment, characterized by excessive accumulation of fibrillar and non-fibrillar collagens, fibronectin and proteoglycans and an influx of fibrotic activators including transforming growth factor (TGF)-β1 (Agostini et al., Proc Am Thorac Soc, 2006, 3:357-363; Border and Noble, N Engl J Med, 1994, 331:1286-1292; Pu et al., Yale J Biol Med. 2013, 86:537-554; Booth et al., Am J Respir Crit Care Med. 2012, 186:866-876; Wynn and Ramalingam, Nat med. 2012, 18:1028-1040). The resulting increase in tissue stiffness impedes proper gas exchange and leads to complete respiratory failure within 2-6 years of diagnosis, unless a lung transplant is performed (Booth et al., Am J Respir Crit Care Med. 2012, 186:866-876; Wynn and Ramalingam, Nat med. 2012, 18:1028-1040; Wynn, J Exp Med. 2011, 208:1339-1350). Considering the poor prognosis of IPF patients, and that one third of patients die on the lung transplant waiting list, significant efforts have been made to develop therapeutics over the past few decades (Williamson et al., Exp Lung Res, 2015, 41:57-73). Recently, pirfenidone and nintedanib have become available as therapeutic treatments for the disease, with both reducing the decline in forced vital capacity (Myllarniemi et al., Eur Clin Respir J, 2015, 2). However, while both treatments ameliorate the decline in lung function, there is currently no curative treatment that reverses the pathology of IPF. The primary difficulties in reversing the characteristic fibrotic damage to the lung are that the pathogenesis of IPF and the mechanisms by which current therapeutics act are poorly defined.

The incomplete understanding of IPF pathogenesis is a result of the unknown origin of lung myofibroblasts, critical effector cells in the transition from a healthy to a mechanically impaired pulmonary tissue (Raghu and Selman, Am J Respir Crit Care Med, 2015, 191:252-254). Classically, resident fibroblasts, epithelial cells and endothelial cells have been considered the primary source of interstitial myofibroblasts, characterized by their enhanced expression of α-smooth muscle actin (SMA) and ability to excessively remodel the extracellular matrix (ECM) (Wynn and Ramalingam, Nat med. 2012, 18:1028-1040; Wynn, J Exp Med. 2011, 208:1339-1350; Hung et al., Am J Respir Crit Care Med, 2013, 188:820-830; Hinz et al., Am J Pathol, 2007, 170:1807-1816). However, recent evidence identifies microvascular pericytes as important contributors to the myofibroblast populations in liver, skin and lung fibrosis, with pericyte derived myofibroblasts depositing excessive collagen I and fibronectin (Hung et al., Am J Respir Crit Care Med, 2013, 188:820-830; Humphreys et al., Semin Nephrol, 2012, 32:463-470; Schrimpf and Duffield, Curr Opin Nephrol Hypertens, 2011, 20:297-305; Rajkumar et al., Arthritis Res Ther, 2005, 7:R1113-1123; Coward et al., Ther Adv Respir Dis, 2010, 4:367-388; Friedman, Nat Clin Pract Gastroenterol Hepatol, 2004, 1:98-105). Genetic fate mapping experiments, using Gli1+ or Foxd1+ progenitors in mouse bleomycin fibrosis models, demonstrated that 37% to 68% of lung myofibroblasts are of microvascular pericyte lineage (Hung et al., Am J Respir Crit Care Med, 2013, 188:820-830; Kramann et al., Cell stem cell, 2015, 16:51-66). However, the key effectors driving PC to myofibroblast differentiation, including the altered matrix composition, increased matrix stiffness, or the biochemical stimuli, are still under investigation and are critical to the understanding of IPF pathogenesis.

In order to investigate the role of PC in IPF progression, a human model must be developed that accurately reproduces the pathogenesis of the disease. Current investigations are limited by models that poorly reflect IPF development, such as the bleomycin induced pulmonary fibrosis model in mice, rats and hamsters. While the in vivo bleomycin model manifests with increased matrix deposition, myofibroblast differentiation, and formation of fibrotic foci, it develops more rapidly than IPF and is autoreversible (Williamson et al., Exp Lung Res, 2015, 41:57-73; Moeller et al., Int J Biochem Cell Biol, 2008, 40:362-382). Consequently, the bleomycin model has been ineffective at predicting therapeutic success, with only two currently approved drugs out of >200 that were effective in vivo (Williamson et al., Exp Lung Res, 2015, 41:57-73; Myllarniemi et al., Eur Clin Respir J, 2015, 2; Moeller et al., Int J Biochem Cell Biol, 2008, 40:362-382). Therefore, there is a need in the art for the development of a realistic, humanized, model of the disease matrix environment, utilizing human cells and tissues from the disease state which will allow for scrutiny of pathway signals that drive fibrotic progression and myofibroblast differentiation, and could provide critical information to test therapeutics. The current invention satisfies this need.

### SUMMARY OF THE INVENTION

The present invention relates to a composition comprising a tunable hydrogel, the hydrogel comprising a polymer component and an ECM component, wherein the hydrogel is formed by the step of employing an amount of a crosslinking agent to achieve a Young's elasticity modulus of the substrate that ranges from 0.1 to 150 kPa. In one embodiment, the hydrogel layer comprises polyacrylamide, and the crosslinking agent comprises bis-acrylamide.

In one embodiment, the ECM component of the tunable hydrogel of the invention is one or more of decellularized, solubilized and conjugated to the hydrogel. In one embodiment, the ECM component is chemically conjugated to the hydrogel. In one embodiment, the ECM component is pericyte derived (PC-ECM).

In one embodiment, the Young's elasticity modulus of the tunable hydrogel of the invention is selected from about less than 1 kPa to about 40 kPa. In one embodiment, the Young's elasticity modulus is selected to mimic a tissue. In one embodiment, a tissue is healthy lung and Young's elasticity modulus is selected to be about 2 kPa. In one embodiment, a tissue is a fibrotic lung and Young's elasticity modulus is selected to be greater than 20 kPa.

The invention further relates to a method of generating a tunable hydrogel, comprising selecting a ratio of polymer to crosslinking agent such that the resulting hydrogel comprises a Young's elasticity modulus selected to mimic a tissue; contacting the selected amount polymer with the selected amount of crosslinking agent in the presence of light and an aqueous solution to form a hydrogel; and contacting the hydrogel with decellularized, solubilized ECM. In one embodiment, the method further comprises setting the elasticity of the substrate by selecting a concentration of cross-linking density within the matrix, such that Young's elasticity modulus is adjustable over several orders of magnitude, from extremely soft to stiff.

In one embodiment, the hydrogel comprises a hydrogel component and an ECM component, wherein the ECM is decellularized, solubilized and conjugated to the hydrogel. In one embodiment, the ECM is PC-ECM.

In one embodiment, the invention relates to a method for culturing an anchorage-dependent cell, comprising: providing a substrate having an elasticity defined by Young's elasticity modulus; introducing the anchorage-dependent cell onto said substrate; and culturing the anchorage-dependent cell. In one embodiment, the substrate comprises a tunable matrix formed by the step of employing an amount of a crosslinker to achieve a finely tunable Young's elasticity modulus of the substrate that ranges from 0.1 to 150 kPa. In one embodiment, the substrate comprises polyacrylamide, and the crosslinker comprises bis-acrylamide.

The invention further relates to a kit comprising the tunable hydrogel of the invention.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts flow cytometry plots of cultured PC isolated from human placental microvessels, demonstrating that PC uniformly express the cell surface molecules NG2, CD90, PDGFR-β, and CD146 and the intracellular protein α-SMA. PC do not express the intracellular proteins SMMHC, a marker expressed by smooth muscle cells, CD31 or CD34, markers expressed by endothelial cells, or CD45, a marker expressed by leukocytes. Specific staining is shown by the bolded black line whereas isotype control is shown in grey. Representative plots are shown for pericyte isolations from N>5 individual human donors.

FIG. 2, comprising FIGS. 2A and 2B, depicts polyacrylamide substrates of varying stiffness, made by altering the relative concentration of crosslinker. Healthy and IPF lung matrices were solubilized and conjugated to the polyacrylamide hydrogels and the mechanical properties of low, medium, and high stiffness hydrogels were measured using an Instron 5848 with a 10N load cell by cross-head displacement. FIG. 2A depicts the Young's Moduli are calculated at 20% strain (* p<0.05 compared to low stiffness hydrogel of the same ECM, #p<0.05 compared to medium stiffness hydrogel of the same ECM, N>5). One-way ANOVA was used. To confirm conjugation of ECM to the polyacrylamide hydrogels, the ECM was tagged with NHS-FITC, dialyzed, and conjugated to the polyacrylamide hydrogel. The excess ECM-FITC was removed by rinsing. FIG. 2B depicts fluorescent images taken for control, 0.2 mg/mL (collagen I and fibronectin), and 0.5 mg/mL of both the healthy and IPF lung ECM.

FIG. 3, comprising FIGS. 3A to 3B, depicts polyacrylamide substrates of varying stiffness made by altering the relative concentration of crosslinker. FIG. 3A depicts representative images of the low, medium and high stiffness hydrogels, shown on a custom made apparatus with a 15 mg sphere placed on the center. FIG. 3B depicts Young's Modulus of low, medium, and high stiffness hydrogels, measured using a modified model of the Hertz equation (* p<0.05 compared to low stiffness hydrogel, #p<0.05 compared to medium stiffness hydrogel, N>4). One-way ANOVA was used.

FIG. 4, comprising FIGS. 4A to 4I, depicts the pericytes to myofibroblast transition present in IPF. Control and IPF lung samples were fixed and stained to observe contribution of PC to myofibroblast population in IPF. FIG. 4A depicts immunofluorescence images of human lung tissue from control and IPF patients. Human lungs are costained for α-smooth muscle actin (α-SMA) with PC marker NG2 and Dapi. Boxes and arrows represent areas of NG2 and α-SMA dual positive costaining in lung tissue. FIG. 4B depicts an exemplary image analysis used to quantify the % coexpression ±SEM of α-SMA+NG2+ cells in control lung samples. FIG. 4C depicts an exemplary image analysis used to quantify the % coexpression ±SEM of α-SMA+NG2+ cells in IPF lung samples. Compared with NHLFs, relative expression of NG2 message is increased in IPF lung fibroblasts (2-tailed Student t test with Bonferroni post-test, ****P<0.001 compared with control lung, n=5). FIG. 4D depicts Masson's Trichrome and picrosirius red images of decellularized lung samples for control and IPF matrix. Mechanical properties of decellularized lung were measured using an Instron5848 with a 10N load by cross-head displacement. FIG. 4E depicts stress-strain plots. FIG. 4F depicts Young's Moduli calculated at 20% strain (* p<0.05 compared to control lung ECM, N>8). FIG. 4G depicts representative images of seven-day PC cultures on decellularized lung scaffolds were stained for α-SMA and IHC on control and IPF ECM. FIG. 4H depicts α-SMA expression presented as mean±SEM using immunoblotting and normalized to GAPDH (black lines used to designate samples run in the same gel but noncontiguous lanes). FIG. 4I depicts MKL1 translocation was examined by extracting nuclear protein for PC cultured on control or IPF lung, was analyzed using immunoblotting, and was presented as mean ±SEM normalized to lamin A/C (2-tailed Student t test with Bonferroni post-test, *P<0.05 compared with control lung, n=3 and 4).

FIG. 5, comprising FIGS. 5A to 5C, depicts an analysis of vimentin expression in decellularized human lung samples from healthy and IPF patients. FIG. 5A depicts vimentin expression in PC cultured on the lung scaffolds for 7 days, quantified using western blot analysis and normalized to GAPDH (N>3, Two-tailed Student t-test). FIG. 5B depicts an analysis of vimentin expression in healthy and IPF lung matrices digested and conjugated to polyacrylamide hydrogels of low, medium and high stiffness. PC were cultured on the hydrogels for 7 days and vimentin expression was quantified using flow cytometry analysis (N>9, one-way ANOVA). FIG. 5C depicts an analysis of vimentin expression after polyacrylamide substrates of low, medium, and high stiffness were functionalized with fibronectin and collagen I. PC were cultured on the hydrogels for 7 days and vimentin expression was quantified using flow cytometry analysis (N>4, one-way ANOVA).

FIG. 6, comprising FIG. 6A through FIG. 6J, depicts experimental results demonstrating that increased substrate stiffness induces megakaryoblastic leukemia 1-dependent (MKL1-dependent) α-SMA expression. Round glass coverslips were coated with polyacrylamide hydrogels with varying stiffness to evaluate the mechanotransductive effects in pericytes. Elasticity of polyacrylamide hydrogels was tested. FIG. 6A depicts exemplary stress-strain plots that were generated. FIG. 6B depicts the Young's Moduli calculated at 20% strain, reported as mean ±SEM (one-way ANOVA with Tukey Post-Hoc test, *P<0.05 versus low-stiffness hydrogel, #P<0.05 versus medium stiffness hydrogel, n=9 and 10). Control and IPF lung protein were conjugated to the polymer, and PC were cultured for 7 days. FIG. 6C depicts immunofluorescence images of α-SMA and F-Actin. FIG. 6D depicts an analysis of the cell area ±SEM for PC. FIG. 6E depicts an analysis of the cell shape (aspect ratio ±SEM) (one-way ANOVA with Tukey Post-Hoc test, *P<0.05 compared with low-stiffness hydrogel of the same matrix, #P<0.05 versus medium stiffness hydrogel of the same matrix, n≥10). FIG. 6F depicts α-SMA expression was quantified using flow cytometry, presented as mean fold increase over expression of cells on low-stiffness healthy lung ±SEM (one-way ANOVA with Tukey Post-Hoc test *P<0.05 compared with low-stiffness hydrogel of the same matrix, n=9). FIG. 6G depicts MKL1 translocation was analyzed using immunoblotting. FIG. 6H depicts MKL1 translocation mean ±SEM normalized to lamin A/C (one-way ANOVA with Tukey Post-Hoc test, *P<0.05 versus low-stiffness hydrogel, n=5). FIG. 6I depicts immunofluorescence images of α-SMA, F-Actin, and nuclei are shown for PC on high-stiffness hydrogels with no treatment (naive) and 0- or 7-day treatment with CCG-1423, an inhibitor of MKL1. FIG. 6J depicts α-SMA expression collected cells was quantified using flow cytometry and presented as fold increase in α-SMA ±SEM compared with untreated cells on low-stiffness healthy lung (one-way ANOVA with Tukey Post-Hoc test, *P<0.05 versus naive PC, n=4-6).

FIG. 7, comprising FIG. 7A through FIG. 7D, depicts results of exemplary experiments demonstrating that varying matrix content does not alter PC to expression of α-SMA. Human fibronectin containing the EDA fragment was isolated from pericyte culture condition media using gelatin affinity chromatography. FIG. 7A depicts exemplary experimental results demonstrating that immunoblotting using an EDA-FN specific antibody (IST-9) confirmed the presence of EDA-FN, but did not bind to plasma fibronectin. Two concentrations of EDA-FN and collagen I (0.1 mg/mL and 0.5 mg/mL) were conjugated to hydrogels of low, medium, and high stiffness. FIG. 7B depicts exemplary experimental results demonstrating an analysis of α-SMA expression from PC cultured on the hydrogels for 7 days and quantified using flow cytometry analysis for hydrogels conjugated to EDA-FN. FIG. 7C depicts exemplary experimental results demonstrating an analysis of α-SMA expression from PC cultured on the hydrogels for 7 days and quantified using flow cytometry analysis for hydrogels conjugated to collagen I. (* p<0.05 compared to low stiffness hydrogel conjugated to 0.1 mg/mL of the corresponding protein, N>3). FIG. 7D depicts exemplary experimental results demonstrating that no significant difference was seen between ECM concentrations. Images of PC on the hydrogels at 7 days are shown. PC were cultured on hydrogels of low, medium, and high stiffness functionalized with a 1:1 ratio of fibronectin and collagen I. (* p<0.05 versus low stiffness hydrogel, #p<0.05 versus medium stiffness hydrogel, N=4).

FIG. 8, comprising FIGS. 8A to 8K, depicts experimental results demonstrating that stiffness induced differentiation is a result of MKL1 translocation. FIG. 8A depicts immunofluorescence images of α-SMA (green) and F-Actin (red) from PC cultured on hydrogels of low, medium, and high stiffness functionalized with fibronectin and collagen I. FIGS. 8B and 8C depict analyses of cell size and shape respectively. FIG. 8D depicts α-SMA expression quantified using flow cytometry. FIG. 8E depicts a confirmation of α-SMA expression using immunoblotting (* p<0.05 versus low stiffness hydrogel, #p<0.05 versus medium stiffness hydrogel, N>4). FIG. 8F depicts MKL1 translocation examined by extracting nuclear protein for PC cultured on control or IPF lung and analyzed using immunoblotting. FIG. 8G depicts similar data to FIG. 8F normalized to lamin A/C (* p<0.05 compared to control lung ECM, N=3, Two-tailed Student t-test). FIG. 8H depicts nuclear protein isolated for PC cultured on the varying stiffness hydrogels and analyzed using immunoblotting. FIG. 8I depicts similar data to FIG. 8H normalized to lamin A/C (* p<0.05 versus low stiffness hydrogel, N>5, one-way ANOVA). FIG. 8J depicts representative immunofluorescence images of α-SMA (green) and F-Actin (red) for PC on high stiffness hydrogels with no treatment (naïve) and early or late treatment with CCG-1423, an inhibitor of MKL1. FIG. 8K depicts α-SMA expression quantified using flow cytometry (* p<0.05 versus naïve PC, N>6, one-way ANOVA).

FIG. 9, comprising FIGS. 9A to 9F, depicts bleomycin activation overcomes stiffness induced myofibroblast transition. FIG. 9A depicts representative immunofluorescence images of F-Actin from PC, cultured on the low, medium, and high stiffness hydrogels, activated with TGF-β1 or bleomycin at 7 days. FIGS. 9B and 9C depict cell size and cell shape of the PC from FIG. 9A respectively (* p<0.05 compared to low stiffness hydrogel, N>5). FIG. 9D depicts immunofluorescence images of α-SMA (green) and vimentin (red) for TGF-β1 or bleomycin activated PC on the varying substrates. FIGS. 9E and 9F depict expression of α-SMA and vimentin myofibroblast markers respectively, quantified using flow cytometry for TGF-β1 or bleomycin activated PC on the varying substrates (* p<0.05 compared to low stiffness hydrogel of the same activation, #p<0.05 compared to non-activated condition of the same hydrogel stiffness, N>3). One-way ANOVA was used.

FIG. 10, comprising FIGS. 10A to 10D, depicts a comparision of non-activated and activated PC. FIG. 10A depicts immunofluorescent images of α-SMA and vimentin from PC cultured on glass coverslips, activated with control media or TGF-β1 for 7 days. FIGS. 10B and 10C depicts α-SMA and vimentin expression respectively, quantified using flow cytometry analysis. FIG. 10D depicts a confirmation of α-SMA and vimentin expression using western blot analysis (N>5). Two-tailed Student t-test was used.

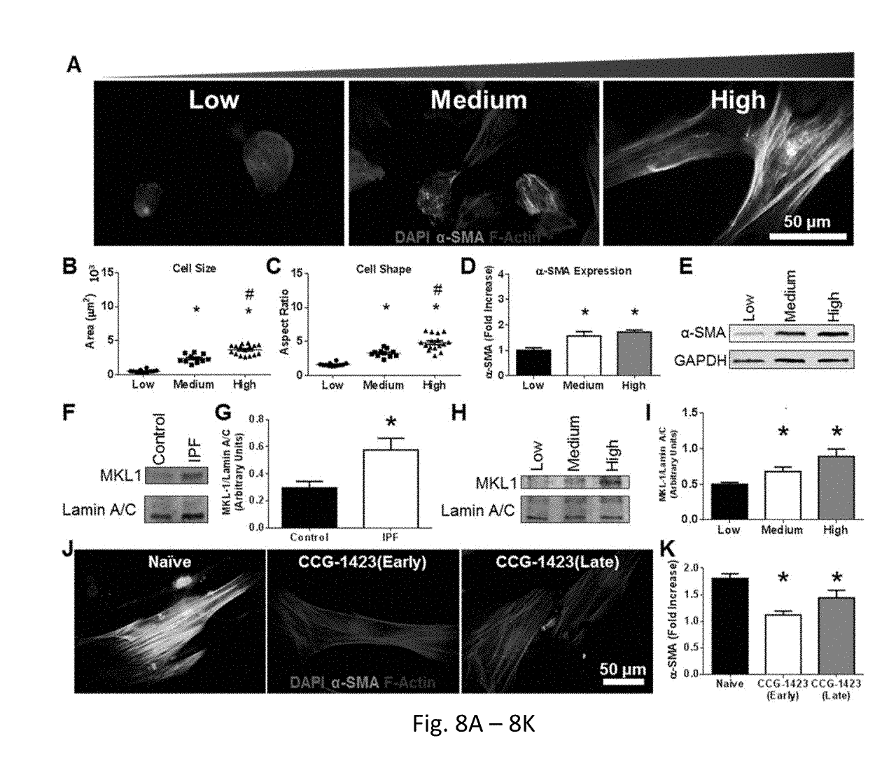

FIG. 11, comprising FIG. 11A through FIG. 11I, depicts exemplary experimental results demonstrating that TGF-β1 promotes extracellular matrix (ECM) remodeling and fibrotic foci formation. PC were cultured on matrices and activated with TGF-β1 for 14 days. FIG. 11A depicts exemplary immunofluorescence images of α-SMA, vimentin, and nuclei and mean expression ±SEM. FIG. 11B depicts exemplary experimental results demonstrating that α-SMA and vimentin were quantified using flow cytometry (one-way ANOVA with Tukey Post-Hoc test, *P<0.05 compared with low-stiffness hydrogel of the same activation, ^{#}P<0.05 compared with nonactivated condition of the same hydrogel stiffness, n=3). FIG. 11C depicts exemplary immunofluorescence images of collagen IV and fibronectin, or collagen I and laminin, of fibrotic lesions. FIG. 11D depicts exemplary experimental results demonstrating the mean spatial density ±SEM of the lesions were quantified. FIG. 11E depicts exemplary experimental results demonstrating the mean area ±SEM of the lesions were quantified (one-way ANOVA with Tukey Post-Hoc test, *P<0.05 versus nonactivated PC, n=6). FIG. 11F depicts exemplary immunofluorescence images of α-SMA and vimentin. FIG. 11G depicts exemplary experimental results demonstrating α-SMA expression quantified using flow cytometry. FIG. 11H depicts exemplary experimental results demonstrating vimentin expression quantified using flow cytometry (mean fold increase over nonactivated α-SMA or vimentin ±SEM shown). FIG. 11I depicts exemplary experimental results demonstrating that expression of α-SMA or vimentin was confirmed using immunoblotting (Student nest with Bonferroni post-test. *P<0.05 versus nonactivated PC, n≥10).

FIG. 12, comprising FIG. 12A through FIG. 12L, depicts experimental results demonstrating that the effects of TGF-β1 induced lung matrix stiffening and increased α-SMA expression are reversed by nintedanib treatment. PC were cultured on glass, activated with TGF-β1 for 28 days, and treated with nintedanib for 7 days. FIG. 12A depicts exemplary immunofluorescence images of collagen I and fibronectin. FIG. 12B depicts a western blot confirming matrix deposition. FIG. 12C depicts exemplary experimental results demonstrating quantification of fibronectin using immunoblotting. FIG. 12D depicts exemplary experimental results demonstrating quantification of collagen I using immunoblotting. FIG. 12E depicts exemplary experimental results demonstrating fibrotic lesions were quantified from images (n≥10) at day 28 (represented as mean ±SEM, Student t test with Bonferroni post-test, *P<0.05 versus TGF-β1). FIG. 12F depicts exemplary experimental results demonstrating α-SMA expression was quantified using flow cytometry (presented as mean fold increase over protein expression of cells cultured on low-stiffness healthy lung ±SEM). FIG. 12G depicts exemplary experimental results demonstrating vimentin expression was quantified using flow cytometry (presented as mean fold increase over protein expression of cells cultured on low-stiffness healthy lung ±SEM).). FIG. 12H depicts exemplary experimental results demonstrating that the expression results were confirmed using immunoblotting (n≥10). Black lines are used to designate samples run in same gel but in noncontiguous lanes. FIG. 12I depicts exemplary experimental results demonstrating that PC were cultured on decellularized control lung with TGF-β1 activation for 28 days and treated with nintedanib for 7 days. Matrix deposition was evaluated by Masson's trichrome and Picrosirius red staining. FIG. 12J depicts exemplary experimental results demonstrating that Mean Young's Moduli ±SEM of the decellularized lung were determined using an Instron5848 at 20% strain (Student nest with Bonferroni post-test, *P<0.05 versus TGF-β1, n=7-9). FIG. 12K depicts exemplary experimental results demonstrating that α-SMA expression was evaluated using immunohistochemistry (IHC). FIG. 12L depicts exemplary experimental results demonstrating that the spatial density of α-SMA⁺ myofibroblasts was quantified (mean ±SEM) from representative α-SMA IHC images (Student t test with Bonferroni post-test, *P<0.05 versus TGF-β1, n=4-6).

FIG. 13, comprising FIG. 13A through FIG. 13G, depicts exemplary experimental results demonstrating that nintedanib induces elevated pericyte (PC) production of metalloproteinases MMP8, MMP9, and MMP13. PC supernatant activated with TGF-β1±nintedanib were evaluated using ELISA. FIG. 13A depicts exemplary experimental results demonstrating an ELISA against MMP2. FIG. 13B depicts exemplary experimental results demonstrating an ELISA against MMP8. FIG. 13C depicts exemplary experimental results demonstrating an ELISA against MMP9. FIG. 13D depicts exemplary experimental results demonstrating an ELISA against MMP13. FIG. 13E depicts exemplary experimental results demonstrating an ELISA against MMP10. For FIG. 13A through FIG. 13E, the data are presented as detected MMP concentration (pg/ml) ±SEM (Student t test with Bonferroni post-test; *P<0.05, **P<0.01 versus TGF-β1; n=4, ***P<0.001). Gelatinase zymography was also used to validate activity of metalloproteinases. FIG. 13F depicts exemplary experimental results demonstrating MMP2 production by PC ±nintedanib treatment. FIG. 13G depicts exemplary experimental results demonstrating MMP9 production by PC ±nintedanib treatment (one-way ANOVA with Tukey post-hoc test, *P<0.05 versus TGF-β1, n=6;**P<0.01).

FIG. 14, comprising FIG. 14A through FIG. 14C, depicts diagrams of the TGF-β1 induced mechanotransduction pathway of α-SMA⁺pericyte (PC) accumulation in pulmonary fibrosis. FIG. 14A depicts a diagram showing that pericyte abundant microvasculature reside in the human lung. FIG. 14B depicts a diagram showing that production of TGF-β1 by fibroblasts and resident macrophages in the interstitial space results in accumulation of α-SMA⁺ PC in the fibrotic foci. FIG. 14C depicts a diagram showing [1] the binding of TGF-β1 to TGF-βR1 and TGF-βR2 on the surface of PC initiates a Smad signaling cascade; [2] Smad2/3 gets phosphorylated and [3] translocates to the cell's nucleus; [4] the accumulation of P-Smad2/3 in the nucleus leads to increased binding to the Smad binding element (SBE), which results in increased transcription of fibrotic genes; [5] increased production of matrix proteins, including collagen I, results in an increased ECM stiffness; [6] focal adhesion and focal adhesion kinases (FAK) binding to the stiffened environment results in [7] increased translocation of megakaryoblastic leukemia 1 (MKL-1), yes-associated protein (YAP) and transcriptional coactivator with PDZ-binding motif (TAZ), resulting in [8] increased α-SMA expression and myofibroblast transition.

FIG. 15, comprising FIG. 15A through FIG. 15F, depicts exemplary experimental results demonstrating the effects of early and late pirfenidone and nintedanib treatment on expression of α-SMA and vimentin. FIG. 15A depicts exemplary experimental results demonstrating expression of α-SMA on day 7 with bleomycin activation. FIG. 15B depicts exemplary experimental results demonstrating expression of vimentin on day 7 with bleomycin activation. FIG. 15C depicts exemplary experimental results demonstrating expression of α-SMA on day 28 with bleomycin activation. FIG. 15D depicts exemplary experimental results demonstrating expression of vimentin on day 28 with bleomycin activation. FIG. 15E depicts exemplary experimental results demonstrating expression of α-SMA on day 28 with TGFβ activation. FIG. 15F depicts exemplary experimental results demonstrating expression of vimentin on day 28 with TGFβ activation.

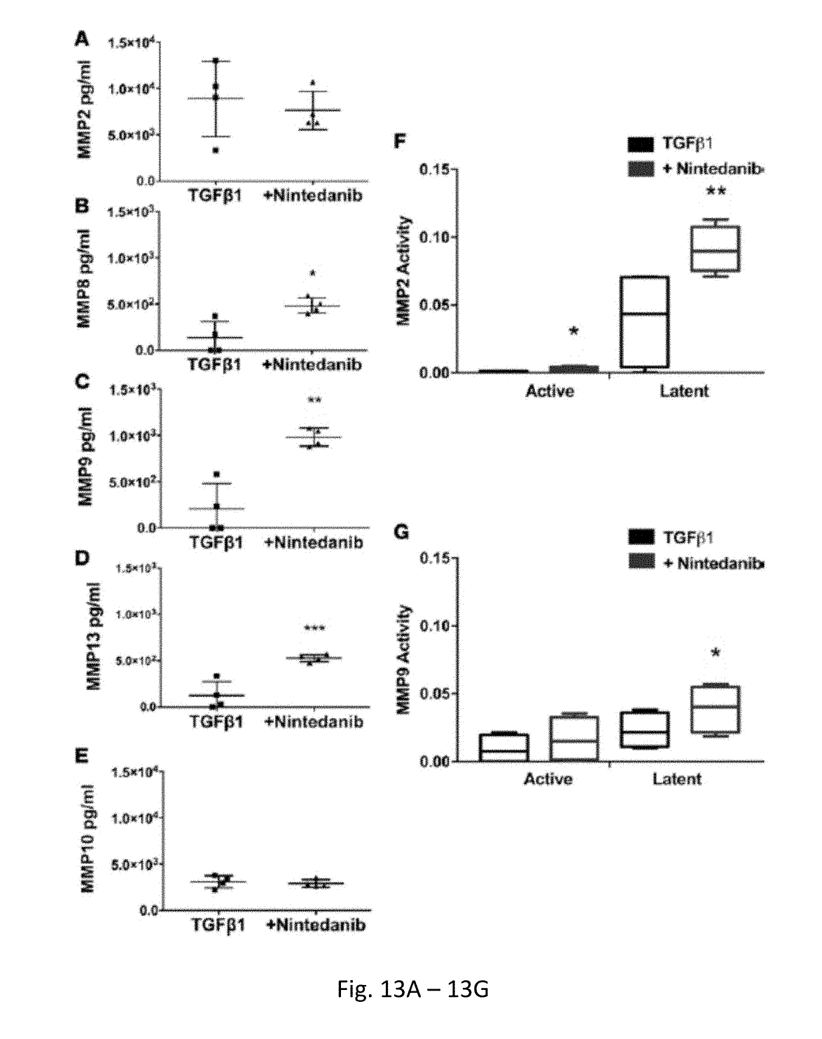

FIG. 16, comprising FIG. 16A through FIG. 16B, depicts exemplary experimental results demonstrating the quantification of collagen I and fibronectin with of early and late nintednib treatment. FIG. 16A depicts exemplary experimental results demonstrating the quantification of collagen I from a western blot. FIG. 16B depicts exemplary experimental results demonstrating the quantification of fibronectin from a western blot.

FIG. 17, comprising FIG. 17A through FIG. 17B, depicts exemplary experimental results demonstrating the effects of early and late nintedanib treatment on expression of α-SMA and vimentin. FIG. 17A depicts exemplary experimental results demonstrating the expression of α-SMA on day 28. FIG. 17B depicts exemplary experimental results demonstrating the expression of vimentin on day 28.

FIG. 18 depicts exemplary experimental results demonstrating the percentage of live cells on day 28 with early and late pirfenidone and nintedanib treatment and TGFβ activation.

FIG. 19 depicts exemplary experimental results demonstrating the number of fibrotic foci following early and late nintedanib treatment and TGFβ activation.

FIG. 20, comprising FIG. 20A through FIG. 20D, depicts exemplary experimental results demonstrating the production of metalloproteinases on day 23 and day 28 following early nintedanib treatment. FIG. 20A depicts exemplary experimental results demonstrating the level of active MMP-2. FIG. 20B depicts exemplary experimental results demonstrating the level of latent MMP-2. FIG. 20C depicts exemplary experimental results demonstrating the level of active MMP-9. FIG. 20D depicts exemplary experimental results demonstrating the level of latent MMP-9.

FIG. 21 depicts exemplary experimental results demonstrating the percentage of live cells on day 28 with early and late nintedanib treatment only.

FIG. 22 depicts exemplary experimental results demonstrating the number of fibrotic lesions following TGFβ activation with or without early nintedanib treatment.

### DETAILED DESCRIPTION

This invention relates to a realistic, humanized model of tissue biomechanical properties (ie, the appropriate matrix composition and mechanical stiffness). The applications for this could be multifold, including creating in vitro models of fibrosis for evaluation of therapeutic intervention of the disease, or substrates for evaluating cell fate (ie, stem cells). An exemplary use of the invention is as a model for idiopathic pulmonary fibrosis (IPF), an incurable interstitial pneumonia of unknown etiology that results in respiratory failure with mortality rates 3-5 years following diagnosis at 50%. The invention provides an improved in vitro model of IPF that captures the hallmark characteristics of the human disease, including myofibroblast differentiation, dense matrix deposition, and increased matrix stiffness would prove beneficial in the evaluation of IPF therapeutics.

The invention provides a composition comprising a ‘tunable’ hydrogel conjugated to decellularized, solubilized, and matrix from a subject. In one embodiment, a subject is healthy. In one embodiment, a subject is a patient having a disease. In one embodiment, a disease is IPF.

In one embodiment, the invention provides an in vitro mimic of the healthy or diseased condition and can be applied to a wide range of diseases that manifest with altered biomechanical properties including fibrosis, chronic obstructive pulmonary disease, diabetes, aging etc. In one embodiment, the invention recreates the native condition in vitro, and can be used to examine disease progression in cells and tissues, or to evaluate the effectiveness of treatments on disease progression. In one embodiment, the disease is fibrosis, and the invention provides a method of evaluating one or more of fibrosis progression and reversal. In one embodiment, the invention provides an in vitro system used to test pharmacological intervention of diseases as well as matrix driven effects on tissue behavior.

The invention relates to mechanically, chemically, and physically tunable (customized) polyacrylamide hydrogel scaffolds that can incorporate extracellular matrix. The extracellular matrix is chemically conjugated to a polyacrylamide hydrogel of tunable mechanical properties and can be used to mimic the native conditions. In some embodiments, the extracellular matrix can be from sources including, but not limited to, healthy human lung and diseased lung tissues.

The invention relates to a polyacrylamide hydrogel with tunable mechanical properties. The elastic modulus of the hydrogels can be tuned, by varying the relative concentration of acrylamide, and its crosslinker, bis-acrylamide, to match that of a tissue. In various embodiments, tissues include, but are not limited to, the brain (<1 kPa), the healthy lung (˜2 kPa), the fibrotic lung (>20 kPa), and non-calcified bone (˜40 kPa).

In one embodiment, extracellular matrix is chemically conjugated to the tunable hydrogel. In one embodiment, the matrix is prepared by decellularization, digestion, and solubilization and then chemically conjugated to the polyacrylamide hydrogels.

The exemplary hydrogel scaffolds are suitable for evaluation of matrix composition and stiffness driven fibrotic progression, and reversal using therapeutic intervention, however the hydrogels of the invention are not limited to modeling of disease conditions and can also be used as a scaffold for cell growth or as a surface to drive cell fate, for example, the invention can be used to induce beneficial cell behavior, such as differentiation of stem cells.

### Definitions

Unless defined otherwise, all technical and scientific terms used herein have the same meaning as commonly understood by one of ordinary skill in the art. Although any methods and materials similar or equivalent to those described herein can be used in the practice or testing of the present invention, the preferred methods and materials are described. As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, or ±5%, or 1%, or ±0.1% from the specified value, as such variations are appropriate.

As used herein, “autologous” refers to a biological material derived from the same individual into whom the material will later be re-introduced.

As used herein, “allogenic” refers to a biological material derived from a genetically different individual of the same species as the individual into whom the material will be introduced.

As used herein, the term “extra-cellular matrix” (or ECM) refers to the extracellular part of animal tissue that provides structural support to the animal cells (in addition to performing various other important functions). The ECM is the defining feature of connective tissue in animals. Proteins commonly found in the ECM include collagen, elastin, fibrin, fibronectin, and laminin (and isoforms thereof). The hydrogels of the present invention may be formed by, for example, exposing collagen or elastin to a polymer. ECM is also important because it provides additional mechanical properties (such as strength and resilience) to the hydrogel, as well as providing an environment that mimics the natural environment.

As used herein, the term “growth medium” is meant to refer to a culture medium that promotes growth of cells. A growth medium will generally contain animal serum. In some instances, the growth medium may not contain animal serum.

The term “polymer”, as used herein, refers to a large molecule (macromolecule) composed of repeating structural units (monomers). These subunits are typically connected by covalent chemical bonds. Polymers can be linear or branched polymers. Preferably, the polymers of the present invention are copolymers comprising three or more different monomers.

The term “proliferation”, as used herein, refers to the reproduction or multiplication of similar forms, especially of cells. That is, proliferation encompasses production of a greater number of cells, and can be measured by, among other things, simply counting the number of cells, measuring incorporation of ³H-thymidine into the cell, and the like.

The term “scaffold”, as used herein, refers to a structure that provides a surface suitable for adherence and proliferation of cells. A scaffold may further provide mechanical stability and support. A scaffold may be in a particular shape or form so as to influence or delimit a tree-dimensional shape or form assumed by a population of proliferating cells. Such shapes or forms include, but are not limited to, films, ribbons, cords, sheets, flat discs, cylinders, spheres, 3-dimensional amorphous shapes, etc.

The term “tissue”, as used herein includes, but is not limited to, bone, brain, neural tissue, fibrous connective tissue including tendons and ligaments, cartilage, dura, pericardia, muscle, lung, heart valves, veins and arteries and other vasculature, dermis, adipose tissue, or glandular tissue.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention includes compositions and methods for the production of tunable hydrogels conjugated to decellularized ECM, for the purpose of mimicking a natural ECM environment, for use in culturing adherent cells. In one embodiment, the tunable ECM conjugated hydrogels of the invention are designed to mimic the ECM environment of organs from large animals, including, for example, humans. In one embodiment, the tunable ECM conjugated hydrogels of the invention are designed to mimic a healthy lung environment. In one embodiment, the tunable ECM conjugated hydrogels of the invention are designed to mimic a diseased lung environment. While the description of the invention is exemplified for the production of lung-mimic ECM, a skilled artisan would recognize that the present invention is not limited to the lung. Rather, the compositions, methods, and kits of the present invention are suitable for mimicking the ECM environment of any biological tissue or organ.

In one embodiment, the present invention provides for the production of small decellularized-ECM hydrogels for use in high-throughput studies. These hydrogels may be used, for example, in studies examining the effects of mechanical, pharmacological, biochemical, and environmental conditions on cells.

The present invention relates to a hydrogel comprising an extra-cellular matrix component and a polymer component. In one embodiment, a polymer of the invention comprises a material that allows for attachment of cells. In one embodiment, a polymer of the invention comprises a material that allows for conjugation to an ECM. In one embodiment, a hydrogel of the invention is comprised of a material that allows for at least a portion of the scaffold to have an elastic modulus in the range of normal human tissue.

In one embodiment, a tissue having a disease has an elastic modulus that is different from healthy tissue of the same type. The hydrogel of the invention is tunable such that, in various embodiments, it provides a biomimetic environment of a diseased or healthy tissue.

### Hydrogel

The hydrogel may comprise any biopolymer or synthetic polymer known in the art. For example, the hydrogel may comprise hyaluronans, chitosans, alginates, collagen, dextran, pectin, carrageenan, polylysine, gelatin or agarose. (see: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of hydrogels based on synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. (see A. S Hoffman, 2002Adv. Drug Del. Rev, 43, 3-12). In one embodiment, the polymer for use in a hydrogel of the invention is polyacrylamide.

In one embodiment the polymer material is placed on a surface, then polymerized, so that the polymer forms a hydrogel. In one embodiment, polymerization is performed by providing a crosslinking agent. Crosslinking of the hydrogel may be performed using any suitable method known in the art. In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N[α.-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NETS) and other bifunctional cross-linking reagents known to those skilled in the art. In certain embodiments, the hydrogel comprises a photo-activated crosslinking agent. In one embodiment, one or more components of the hydrogel is cross-linked upon exposure to ambient light. In one embodiment, one or more components of the hydrogel is cross-linked upon exposure to UV light. In one embodiment, a crosslinking agent is bis-acrylamide.

In one embodiment the ratio of crosslinking agent to polymer is selected such that the elastic modulus of the resulting scaffold is similar to a tissue. As described herein, in one embodiment, the present invention demonstrates that the elastic modulus of the hydrogels can be tuned by varying the relative concentration of acrylamide, and its crosslinker, bis-acrylamide, to match that of a tissue. In one embodiment, a tissue is healthy lung, and the elastic modulus of the hydrogel is about 2 kPa (4% acrylamide, 0.1% bis-acrylamide, in water). In one embodiment, a tissue is fibrotic lung, and the elastic modulus of the hydrogel is greater than 20 kPa (10% acrylamide, 0.225% bis-acrylamide, in water). In one embodiment, a tissue is brain tissue, and the elastic modulus of the hydrogel is less than 1 kPa (3% acrylamide, 0.06% bis-acrylamide, in water). In one embodiment, a tissue is heart, and the elastic modulus of the hydrogel is about 5 kPa (5% acrylamide, 0.15% bis-acrylamide, in water). In one embodiment, a tissue is kidney, and the elastic modulus of the hydrogel is about 3 kPa (4% acrylamide, 0.3% bis-acrylamide, in water). In one embodiment, a tissue is liver, and the elastic modulus of the hydrogel is about 3 kPa (4% acrylamide, 0.3% bis-acrylamide, in water). In one embodiment, a tissue is skeletal muscle, and the elastic modulus of the hydrogel is about 11 kPa (10% acrylamide, 0.1% bis-acrylamide, in water). In one embodiment, a tissue is skin, and the elastic modulus of the hydrogel is about 7 kPa (10% acrylamide, 0.06% bis-acrylamide, in water). In one embodiment, a tissue is non-calcified bone, and the elastic modulus of the hydrogel is about 40 kPa (8% acrylamide, 0.48% bis-acrylamide, in water). In various embodiments, the elastic modulus of the hydrogel is selected to be from about 0.1 kPa to about 150 kPa. In various embodiments, the elastic modulus of the hydrogel is selected to be from less than 1 kPa to greater than 40 kPa.

In general, the amount of polymer in the composition of the present invention is an amount that allows for the formation of hydrogels in accordance with the present invention. In some embodiments, the amount of polymer in the composition of the present invention ranges between about 1% w/w and about 90% w/w, between about 2% w/w and about 80% w/w or between about 3% w/w and about 70% w/w. In some embodiments, the amount of polymer is about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w or more.

In general, the amount of cross-linker in the composition of the present invention is an amount that allows for the formation of hydrogels in accordance with the present invention. In some embodiments, the amount of cross-linker in the composition of the present invention ranges between about 0.01% w/w and about 9% w/w, between about 0.02% w/w and about 8% w/w or between about 0.03% w/w and about 7% w/w. In some embodiments, the amount of cross-linker is about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, about 0.10% w/w, about 0.15% w/w, about 0.20% w/w, about 0.25% w/w, about 0.30% w/w, about 0.35% w/w, about 0.40% w/w, about 0.45% w/w, about 0.50% w/w, about 0.55% w/w, about 0.60% w/w, about 0.65% w/w, about 0.70% w/w, about 0.75% w/w, about 0.80% w/w or more. In some embodiments, the amount of cross-linker is approximately 0.225% w/w. As a general rule, the elastic modulus of the hydrogel increases with higher cross-linker concentrations in the composition.

### Additional Molecules

In one embodiment, a scaffold of the invention comprises one or more additional molecules. In one embodiment, additional molecules promote one or more of cell growth or cell differentiation. In one embodiment, an additional molecule is a pharmaceutical agent. Biologically active agents or drug compounds that may be added to the composition and/or hydrogel of the present invention include proteins, glycosaminoglycans, carbohydrates, nucleic acids and inorganic and organic biologically active compounds, such as enzymes, antibiotics, anti-neoplastic agents, local anesthetics, hormones, angiogenic agents, anti-angiogenic agents, growth factors (e.g. insulin-like growth factor-1 (IGF-I), basic fibroblast growth factor (bFGF) and transforming growth factor-b (TGFβ)), antibodies, neurotransmitters, psychoactive drugs, anticancer drugs, chemotherapeutic drugs, drugs affecting reproductive organs, genes, and oligonucleotides.

### Source of ECM

The native ECM is a non-covalent three-dimensional network of proteins and polysaccharides bound together with cells intermixed. The present invention provides a biomimetic three-dimensional environment by adding decellularized ECM molecules onto a hydrogel having an elastic modulus selected to mimic a tissue. When the scaffold of the present invention is functionalized with ECM molecules, it provides support and guidance for cell morphology and tissue development.

In one embodiment, the invention provides a hydrogel conjugated to an ECM material. Tissue serving as the ECM of the invention can be obtained directly from a donor, from a culture of tissue from a donor, from recombinant sources or from established tissue culture lines.

In one embodiment, tissue is from a patient. Following tissue harvest, the tissue is processed to kill the living cells. Care is taken to preserve the extracellular matrix. Guidelines for processing the harvested tissue as described are well known to those skilled in the art. For example, the tissue could be frozen and thawed. In one embodiment, tissue serving as the ECM is obtained directly from a donor, decellularized, solubilized and conjugated to the hydrogel.

In one embodiment, ECM of the invention is synthetic. In one embodiment, tissue is from a healthy donor. In one embodiment, a tissue may be from a recently deceased donor. Guidelines for tissue procurement including surgical technique of removal, number of hours between death of the donor and tissue procurement, and testing of the donor for infectious disease, are well described. In one embodiment, the ECM material can be derived from organ. In one embodiment, an organ can be one or more of brain, liver, skin, kidney, heart, skeletal muscle, and lung. An organ can be a healthy organ or a diseased organ. ECM derived from an organ can be obtained from a donor, from a culture of tissue from a donor or from cells derived from an organ (e.g. lung cells).

In one embodiment, the ECM is pericyte-derived ECM (PC-ECM). PC-ECM is particularly useful in applications where animal models are not available or not physiologically relevant. Methods of generating PC-ECM are known in the art and include the general steps of: isolating PC from human placental microvascular segments, culturing PC using methods known in the art (e.g., in M199 (Gibco, Grand Island, N.Y., USA) supplemented with fetal bovine serum), and activating the PC culture with cytokines (e.g., TFG-B1, IL-1B, and CCL2). Following several weeks of culturing, the PC cellular layer can be removed. In one embodiment, PC-ECM is decellularized, solubilized and conjugated to the hydrogel.

In one embodiment, the ECM is conjugated to the hydrogel. In one embodiment, the ECM is chemically conjugated to the hydrogel. A crosslinker used for chemically conjugating the ECM to the hydrogel may be, but is not limited to, one of sulfo-SANPAH (sulfosuccinimidyl 6-(4′-azido-2′-nitrophenylamino)hexanoate); DTME (dithiobismaleimidoethane); BM(PEG)2 (1,8-bismaleimido-diethyleneglycol); Sulfo-NHS (N-hydroxysulfosuccinimide); BM(PEG)3 (1,11-bismaleimido-triethyleneglycol); BS3 (bis(sulfosuccinimidyl)suberate); SM(PEG)n (PEGylated SMCC crosslinker, with n being an integer between and including 2 to 24); SBAP (succinimidyl 3-(bromoacetamido)propionate); DCC (dicyclohexylcarbodiimide); EDC (1-ethyl-3-(3-dimethylaminopropyl)carbodiimide hydrochloride); BS(PEG)n (PEGylated bis(sulfosuccinimidyl)suberate, with n being an integer between and including 2 to 24); EDC (1-ethyl-3-(3-dimethylaminopropyl)carbodiimide hydrochloride); Sulfo-KMUS (N-κ-maleimidoundecanoyl-oxysulfosuccinimide ester); BS2G-d0 (bis(sulfosuccinimidyl) glutarate-d0); EGS (ethylene glycol bis(succinimidyl succinate)); DSP (dithiobis(succinimidyl propionate)); BMPH (N-β-maleimidopropionic acid hydrazide); NHS-Phosphine; Sulfo-MBS (m-maleimidobenzoyl-N-hydroxysulfosuccinimide ester); PDPH (3-(2-pyridyldithio)propionyl hydrazide); DMA (dimethyl adipimidate); BS2G-d4 (bis(sulfosuccinimidyl) 2,2,4,4-glutarate-d4); Sulfo-EGS (ethylene glycol bis(sulfosuccinimidyl succinate)); L-Photo-Methionine; BMPS (N-β-maleimidopropyl-oxysuccinimide ester); N-(t-BOC)-Aminooxyacetic Acid, Tetrafluorophenyl Ester; PMPI (p-maleimidophenyl isocyanate); SMPH (Succinimidyl 6-((beta-maleimidopropionamido)hexanoate)); DMP (dimethyl pimelimidate); Sulfo-SMCC (sulfosuccinimidyl 4-(N-maleimidomethyl)cyclohexane-1-carboxylate); DTBP (dimethyl 3,3′-dithiobispropionimidate); EDAC, 1-Ethyl-3-(3-Dimethylaminopropyl)carbodiimide, Hydrochloride; PEG4-SPDP (PEGylated, long-chain SPDP crosslinker); SPDP (succinimidyl 3-(2-pyridyldithio)propionate); LC-SPDP (succinimidyl 6-(3(2-pyridyldithio)propionamido)hexanoate); SDA (NHS-Diazirine) (succinimidyl 4,4′-azipentanoate); BMOE (bismaleimidoethane); Sulfo-LC-SDA (Sulfo-NHS-LC-Diazirine) (sulfosuccinimidyl 6-(4,4′-azipentanamido)hexanoate); SPB (succinimidyl44-(psoralen-8-yloxy)]-butyrate); SDAD (NHS-SS-Diazirine) (succinimidyl 2-((4,4′-azipentanamido)ethyl)-1,3′-dithiopropionate); MPBH (4-(4-N-maleimidophenyl)butyric acid hydrazide); BSOCOES (bis(2-(succinimidooxycarbonyloxy)ethyl)sulfone); Sulfo-SIAB (sulfosuccinimidyl (4-iodoacetyl)aminobenzoate); DMS (dimethyl suberimidate); AMAS (N-α-maleimidoacet-oxysuccinimide ester); SIA (succinimidyl iodoacetate); ATFB, SE, 4-Azido-2,3,5,6-Tetrafluorobenzoic Acid, Succinimidyl Ester; DTSSP (3,3′-dithiobis(sulfosuccinimidyl propionate)); DFDNB (1,5-difluoro-2,4-dinitrobenzene); DSG (disuccinimidyl glutarate) and DST (disuccinimidyl tartrate).

In general, the amount of decellularized, solubilized ECM in the composition of the present invention is an amount that allows for the formation of hydrogels in accordance with the present invention. In some embodiments, the amount of ECM in the composition of the present invention ranges between about 1% w/w and about 60% w/w, between about 1% w/w and about 50% w/w, between about 1% w/w and about 40% w/w, between about 5% w/w and about 30% w/w, between about 5% w/w and about 20% w/w, or between about 5% w/w and about 10% w/w. In some embodiments, the percent of ECM is about 1% w/w, about 2% w/w, about 3% w/w, about 4% w/w, about 5% w/w, about 6% w/w, about 7% w/w, about 8% w/w, about 9% w/w, about 10% w/w, about 20% w/w, about 30% w/w, about 40% w/w, about 50% w/w, or more.

### Methods of Using the Tunable ECM-Conjugated Hydrogels

In one embodiment, the tunable ECM-conjugated hydrogels of the invention are used to evaluate the effect of a treatment on cells or as a cell culture substrate. The tunable ECM-conjugated hydrogels is typically seeded with the cells; the cells are cultured; and then the cells are assayed. In one embodiment, cells are optionally treated and the effect of the treatment is assayed. In one embodiment, a treatment is a pharmaceutical agent. In one embodiment, a treatment serves to induce cellular differentiation.

### Cells

The hydrogel of the present invention may also include cells either encapsulated in the hydrogel or attached to the surface of the hydrogel.

In general, cells to be used in accordance with the present invention are any types of cells. The cells should be viable when encapsulated within the hydrogels of the present invention or when adhered to the surface of the hydrogel. In some embodiments, cells appropriate for use with the hydrogels in accordance with the present invention include, but are not limited to, mammalian cells (e.g. human cells, primate cells, mammalian cells, rodent cells, etc.), avian cells, fish cells, insect cells, plant cells, fungal cells, -bacterial cells, and hybrid cells. In some embodiments, exemplary cells that can be encapsulated within hydrogels include stem cells, totipotent cells, pluripotent cells, and/or embryonic stem cells. In some embodiments, exemplary cells that can be encapsulated within hydrogels in accordance with the present invention include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, fibrocytes etc.), ameloblasts, fibroblasts, vascular smooth muscle cells, pericytes, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, may be encapsulated within hydrogels in accordance with the present invention.

Exemplary mammalian cells that can be used in accordance with the present invention include, but are not limited to, human placental cells, Chinese hamster ovary (CHO) cells, HeLa cells, Madin-Darby canine kidney (MDCK) cells, baby hamster kidney (BHK cells), NS0 cells, MCF-7 cells, MDA-MB-38 cells, U87 cells, A172 cells, HL60 cells, A549 cells, SP10 cells, DOX cells, DG44 cells, HEK 293 cells, SHSY5Y, Jurkat cells, BCP-1 cells, COS cells, Vero cells, GH3 cells, 9L cells, 3T3 cells, MC3T3 cells, C3H-10T1/2 cells, NIH-3T3 cells, and C6/36 cells, iPS cells, and embryonic stem cells. Those skilled in the art will recognize that the cells listed herein represent an exemplary, not comprehensive, list of cells that can be utilized in accordance with the present invention.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, cells are layered within a hydrogel. In some embodiments, the hydrogel contains different cell types.

In some embodiments, the conditions under which cells are encapsulated within hydrogels are altered in order to maximize cell viability. In some embodiments, for example, cell viability increases with a higher or lower elastic modulus of the hydrogel. In some embodiments, cells located at the periphery of a hydrogel tend to have decreased viability relative to cells that are fully-encapsulated within the hydrogel. In some embodiments, conditions (e.g. pH, ionic strength, nutrient availability, temperature, oxygen availability, osmolarity, etc) of the surrounding environment may need to be regulated and/or altered to maximize cell viability.

In some embodiments, cell viability can be measured by monitoring one of many indicators of cell viability. In some embodiments, indicators of cell viability include, but are not limited to, intracellular esterase activity, plasma membrane integrity, metabolic activity, gene expression, and protein expression. To give but one example, when cells are exposed to a fluorogenic esterase substrate (e.g. calcein AM), live cells fluoresce green as a result of intracellular esterase activity that hydrolyzes the esterase substrate to a green fluorescent product. To give another example, when cells are exposed to a fluorescent nucleic acid stain (e.g. ethidium homodimer-1), dead cells fluoresce red because their plasma membranes are compromised and, therefore, permeable to the high-affinity nucleic acid stain.

In general, the amount of cells in a composition is an amount that allows for the formation of hydrogels in accordance with the present invention. In some embodiments, the amount of cells that is suitable for forming hydrogels in accordance with the present invention ranges between about 0.1% w/w and about 80% w/w, between about 1.0% w/w and about 50% w/w, between about 1.0% w/w and about 40% w/w, between about 1.0% w/w and about 30% w/w, between about 1.0% w/w and about 20% w/w, between about 1.0% w/w and about 10% w/w, between about 5.0% w/w and about 20% w/w, or between about 5.0% w/w and about 10% w/w. In some embodiments, the amount of cells in a composition that is suitable for forming hydrogels in accordance with the present invention is approximately 5% w/w. In some embodiments, the concentration of cells in a precursor solution that is suitable for forming hydrogels in accordance with the invention ranges between about 10 and about 1×10⁸ cells/mL, between about 100 and about 1×10⁷ cells/mL, between about 1×10³ and about 1×10⁶ cells/mL, or between about 1×10⁴ and about 1×10⁵ cells/mL In some embodiments, a single hydrogel comprises a population of identical cells and/or cell types. In some embodiments, a single hydrogel comprises a population of cells and/or cell types that are not identical. In some embodiments, a single hydrogel may comprise at least two different types of cells. In some embodiments, a single hydrogel may comprise 3, 4, 5, 10, or more types of cells. To give but one example, in some embodiments, a single hydrogel may comprise only embryonic stem cells. In some embodiments, a single hydrogel may comprise both embryonic stem cells and hematopoietic stem cells.

### Media

Any of a variety of cell culture media, including complex media and/or serum-free culture media, that are capable of supporting growth of the one or more cell types or cell lines may be used to grow and/or maintain cells. Typically, a cell culture medium contains a buffer, salts, energy source, amino acids (e.g., natural amino acids, non-natural amino acids, etc), vitamins, and/or trace elements. Cell culture media, may optionally contain a variety of other ingredients, including but not limited to, carbon sources (e.g., natural sugars, non-natural sugars, etc), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites. Cell culture media suitable for use in accordance with the present invention are commercially available from a variety of sources, e.g., ATCC (Manassas, Va.). In certain embodiments, one or more of the following media are used to grow cells: RPMI-1640 Medium, Dulbecco's Modified Eagle's Medium, Minimum Essential Medium Eagle, F-12K Medium., Iscove's Modified Dulbecco's Medium.

### Kit

In one embodiment, the present invention provides a kit comprising one or more pre-formed ECM-conjugated hydrogels. In one embodiment, the present invention provides a kit for the production of one or more tunable ECM-conjugated hydrogel. In one embodiment, the kit of the invention comprises all of the reagents necessary to produce one or more tunable ECM-conjugated hydrogel. In one embodiment, the kit provides decellularized, solubilized ECM for use in producing a tunable ECM-conjugated hydrogel. In one embodiment, the kit comprises reagents for the decellularization and solubalization of ECM. In one embodiment, the kit comprises instructional material which describes, for example, the formation of tunable ECM-conjugated hydrogel constructs, described elsewhere herein.

In one embodiment, the kit of the invention comprises the reagents required for the decellularization of isolated tissue. Various decellularization methods are well known in the art (Daly et al., 2011, Tissue Engineering Part A, 18: 1-16; Wallis et al., 2012, Tissue Engineering Part C: Methods, 18:420-432; Bonenfant et al, Biomaterials; Sokocevic et al, Biomaterials), and thus the kit of the invention comprises reagents required to carry out one or more of these methods.

For example, in certain embodiments, decellularization of an isolated tissue comprises the application and/or perfusion of one or more solutions to the isolated tissue. Therefore, in one embodiment, the kit of the invention comprises one or more solutions, or reagents necessary to make such solutions, to be applied to the isolated tissue in order to decellularize the tissue. In one embodiment, the reagent or solution comprises an ionic detergent, including but not limited to sodium deoxycholate (SDC), sodium dodecyl sulfate (SDS), Triton X-200, and the like. In one embodiment, the reagent or solution comprises a non-ionic detergent, including but not limited to Triton X-100, Triton X-114, and the like. In one embodiment, the reagent or solution comprises an acidic or a basic solution, including but not limited to paracetic acid. In one embodiment, the reagent or solution comprises a hypotonic or hypertonic solution which lyses cells by osmotic pressure. In one embodiment, the reagent or solution comprises a zwitterionic solution, including but not limited to CHAPS, Sulfobetaine-10 and -16, and the like. In one embodiment, the reagent or solution comprises a solvent, including but not limited to alcohols, acetone, or tributyle phosphate. In one embodiment, the reagent or solution comprises enzymes which degrade cellular components or other biomolecules. In certain embodiments, the reagent or solution comprises an enzyme activating element, such as magnesium or calcium. Such enzymes include but are not limited to trypsin, DNase, RNase, dipase, and the like. In one embodiment, the reagent or solutions may be combined with a physical or mechanical method, including but not limited to freezing and thawing, electrical stimulation, physical force, perfusion, sonication, or agitation.

In one embodiment, the kit of the invention comprises the polymer and crosslinking reagents necessary to form a ECM-conjugated hydrogel with a desired elasticity. In one embodiment, the reagents of the kit include, but is not limited to, acrylamide, bis-acrylamide, water, and the like. In some embodiments, the reagents may be applied to decellularized, solubilized ECM as a single solution. In other embodiments, the coating reagents may be provided and applied to decellularized, solubilized ECM as two solutions. For example, the first solution may include the acrylamide, and may be applied to the decellularized ECM. Then, the second solution, containing the crosslinking agent such as bis-acrylamide, may be applied to crosslink the previously applied first solution.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Regulation of Human Microvascular Pericyte to Myofibroblast Transition in Pulmonary Fibrosis

Human-placental derived pericytes, expressing PC markers neural/glial antigen (NG)2, CD90, CD146, and platelet-derived growth factor receptor (PDGFR)-β, while lacking markers for smooth muscle cells, endothelial cells, and leukocytes, respond to pro-inflammatory and pro-fibrotic activation and alter ECM deposition (Sava et al., Microcirculation, 2015, 22:54-67; Brown et al., Cell Mol Bioeng, 2015, 8:349-363; Ayres-Sander et al., PLoS One, 2013, 8:e60025; Lauridsen et al., FASEB, 2014, 28:1166-1180; Maier et al., Microcirculation, 2010, 17:367-380). Here, the mechanistic drivers of PC contribution to fibrosis have been identified by investigating the ability of pericytes to differentiate into a myofibroblast lineage in response to altered matrix composition, substrate stiffness, and biochemical stimuli. Native human lung tissue was used to create a novel human lung conjugated polymer hydrogel, allowing for decoupling of the effects of the altered ECM composition and mechanical properties of the fibrotic lung on PC to myofibroblast differentiation. Overall, this represents the first instance of chemically conjugating ECM from normal and IPF lungs to a mechanically tunable hydrogel system, allowing for mimicking or altering the native environment. The effects of TGF-β1 on PC remodeling of the human lung and differentiation into myofibroblast (Harrison and Lazo, J Pharmacol Exp Ther, 1987, 243:1185-1194; Phan et al., J Clin Invest, 1985, 76:241-247), and the use of nintedanib, as well as CCG-1423, to prevent or reverse PC to myofibroblast transition (Myllarniemi et al., Eur Clin Respir J, 2015, 2; Evelyn et al., Mol Cancer Ther, 2007, 6:2249-2260) were evaluated utilizing the ECM-conjugated hydrogel substrate.

The tunable ECM-conjugated hydrogel substrate offers a mechanism for further studies examining the independent and synergistic contributions of matrix composition and substrate stiffness to cellular behavior as well as a screening tool for future investigations of disease and associated therapeutics. The novel methods and results derived from this work will advance the understanding of pathogenesis and progression in human disease. Further, the results demonstrate that the PC are capable of differentiation into myofibroblasts and phenotypic reversal via mechanosensing of the microenvironment. Equally as important, the results demonstrate that therapeutic strategies targeted towards inhibition of cellular mechanotransduction are sufficient to halt the progression of PC pro-fibrotic function and induce the reversal of PC pro-fibrotic phenotype.

The materials and methods are now described.

### Materials and Methods

Preparation of Human Lung

Explanted human lung tissue, from the right and left lower lung, were obtained from Prof. Erica Herzog (Yale University). For immunofluorescence evaluation, samples were fixed in 4% paraformaldehyde, blocked with 2% BSA, and stained against NG2 (Clone H-300, Santa Cruz Biotechnology, Santa Cruz, Calif., USA), CD90 (Clone K-15, Santa Cruz), PDGFR-β (Clone 18A2, Santa Cruz), or α-SMA (Clone 1A4, Santa Cruz).

Lung sections were decellularized as previously described (Sun et al., Am J Physiol Lung Cell Mol Physiol, 2014, 306:L463-475; Petersen et al., Science, 2010, 329:538-541). Briefly, the lung pieces were snap frozen, sliced to 2 mm thickness, and rinsed with PBS containing sodium nitroprusside (SNP). The slices were decellularized with 8 mM CHAPS (Sigma-Aldrich, St Louis, Mo., USA), 1M NaCl, and 25 mM EDTA at 37° C. overnight. The slices were treated with 90 U/mL benzonase nuclease (Sigma) for 1 hour and stored in PBS containing 10% penicillin/streptomycin and 2% Gentamicin at 4° C. until the time of culture. For solubilization, the tissue slices were rinsed with PBS, frozen, and lyophilized. The lyophilized pieces were ground to a fine powder using a tissue grinder and digested with 1 mg/mL pepsin (Sigma) in 0.1 M HCl for 72 hours (DeQuach et al., PLoS One, 2010, 5:e13039).

Histological Evaluation

Samples were fixed in 4% paraformaldehyde for 1 hour, dehydrated, embedded in paraffin, and sectioned into 5μm thick slices. Sections were stained with Masson's Trichrome, picrosirius red, and α-smooth muscle actin (SMA) by the Yale Histology Core Facility.

Pericyte Isolation and Culture

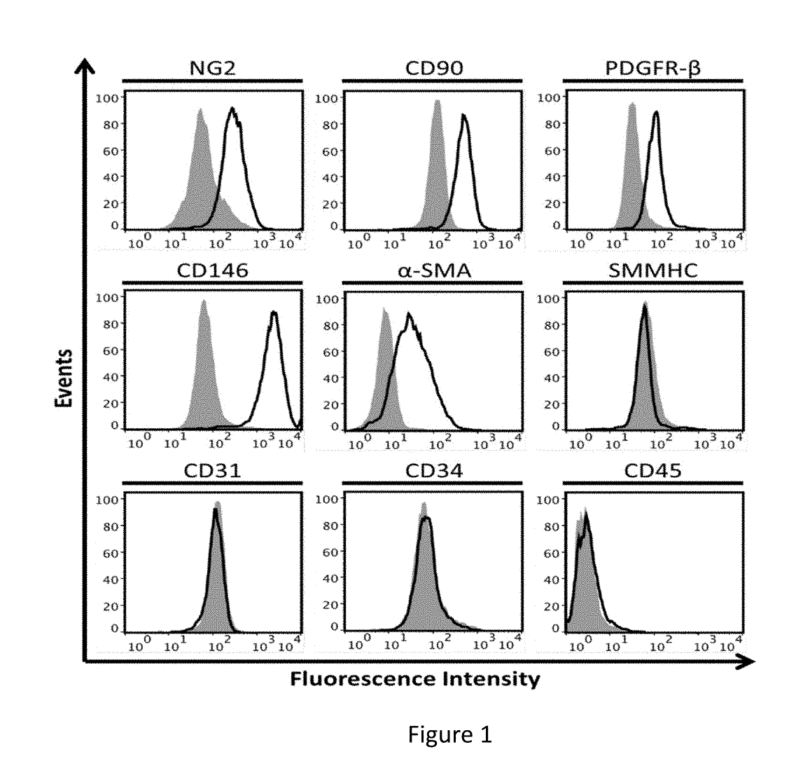

Human microvascular PC were isolated from a readily available and nearly inexhaustible source, human placental tissue, by microvascular segment explant outgrowth (Sava et al., Microcirculation, 2015, 22:54-67; Ayres-Sander et al., PLoS One, 2013, 8:e60025; Lauridsen et al., FASEB, 2014, 28:1166-1180; Maier et al., Microcirculation, 2010, 17:367-380). Human placental tissue was segmented, digested with collagenase, and the resulting cells were cultured in M199 (Gibco, Grand Island, NY, USA) medium supplemented with 20% FBS (Hyclone Laboratories, Logan, Utah, USA), and 1% penicillin-streptomycin (Gibco). Isolated pericytes were screened by flow cytometry against NG2 (H-300, Santa Cruz Biotechnology), CD90 (K-15, Santa Cruz), CD146 (P1H12, Biolegend, San Diego, Calif., USA), PDGFR-β (18A2, Santa Cruz), α-SMA (1A4, Santa Cruz), SMMHC (N-16, Santa Cruz), CD31 (C20, Santa Cruz), CD34 (581, Sigma) and CD45 (MEM-28, Sigma). As negative control, PC were incubated with isotype matched IgG. Samples were run on a Stratedigm S1000EXi Flow Cytometer (Stratedigm, San Jose, Calif. USA) and analyzed with FlowJo software (Tree Star, Ashland, Oreg., USA). FIG. 1 shows representative histograms.

Isolation of EDA Fibronectin

Fibronectin was isolated from pericyte culture media using gelatin affinity chromatography (Ramanathan et al., Biochem Biophys Res Commun, 2014, 443:395-399; Zhang et al, Biotechnol Prog, 2013, 29:493-504). Pericyte culture media was passed through a gelatin separopore column (Bioworld, Dublin, Ohio) and the bound fibronectin was eluted with 6M urea (Sigma). Fibronectin purity was confirmed by sodium dodecyl sulfate polyacrylamide electrophoresis and immmunoblotted with antibody against EDA fibronectin (IST-9, Santa Cruz).

Polyacrylamide Hydrogel Preparation

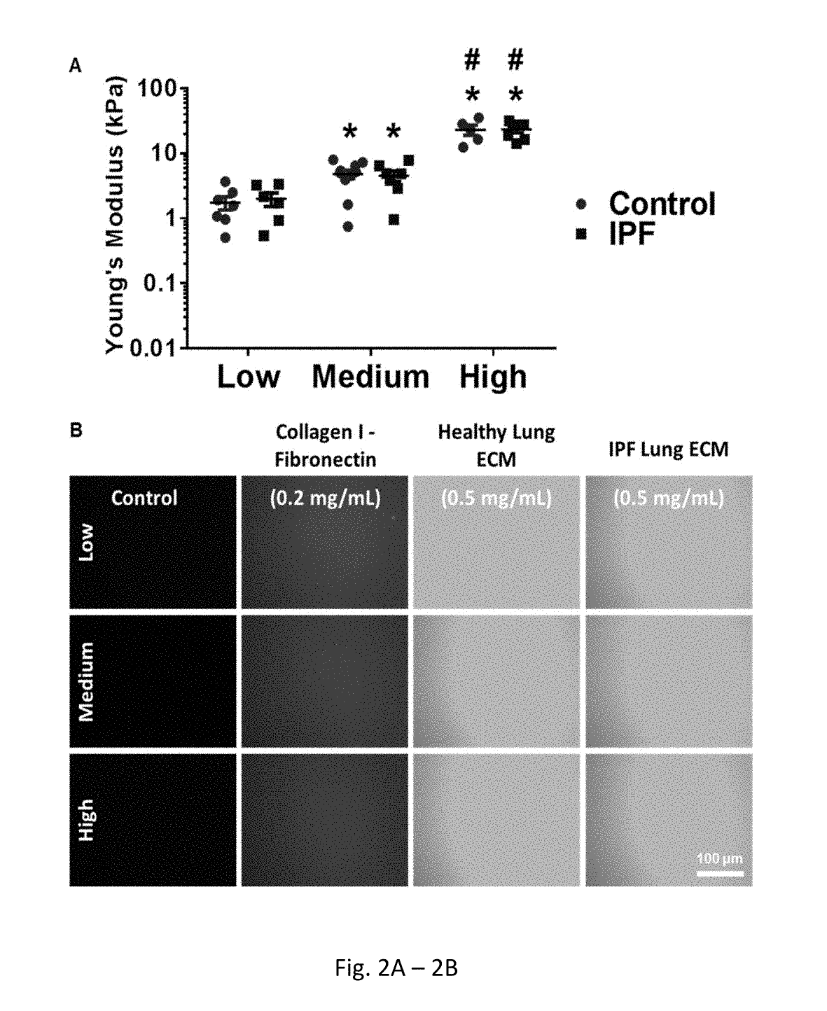

Polyacrylamide hydrogels were prepared as previously described (Tse and Engler, Current protocols in cell biology, editorial board, Juan S. Bonifacino et al, 2010, Chapter 10, Unit 10-16). Briefly, amino-silanated coverslips were prepared by treatment with 0.1M NaOH, followed by 3-aminopropyltriethoxysilane (Sigma), and crosslinked with glutaraldehyde. Chloro-silanated coverslips were treated with dichlorodimethylsilane (Sigma). Polyacrylamide solutions of varying stiffness were prepared by altering the concentrations of acrylamide (Bio-Rad, Hercules, Calif., USA) and bis-acrylamide (Bio-Rad). Ammonium persulfate (Sigma) and tetramethylethylenediamine (Bio-Rad) were used as cross-linkers. The solution was placed between the chloro-silanated and amino-silanated coverslips and cross-linked under ambient lighting for 30 minutes, after which the amino-silanated slide, containing the hydrogel, was hydrated in PBS. The ECM components were chemically conjugated to the polyacrylamide hydrogel using the naturally occurring amine residues on the proteins. Sulfa-SANPAH (Thermo Fisher, Grand Island, N.Y., USA) was added in 50 mM 4-(2-hydroxyethyl)-1-piperazineethanesulfonic acid buffer (Sigma) and the hydrogels were placed under UV light (365 nm, 10 mW/cm²) for 15 minutes, and incubated overnight with either 0.5 mg/mL solubilized healthy or IPF human lung ECM, or isolated ECM proteins (0.1 mg/mL human plasma fibronectin (Millipore, Billerica, Mass., USA) and 0.1 mg/mL collagen I (Sigma). Validation of protein conjugation was performed by tagging ECM with NHS-Fluorescein (5/6-carboxyfluorescein succinimidyl ester), dialyzing overnight, and rinsed extensively (Syed et al., J Vis, Exp, 2015, 97). Immunofluorescence images of tagged proteins are shown in FIG. 2.

Tensile Testing

Decellularized lung samples from control and IPF subjects, as well as polyacrylamide hydrogels of varying crosslinking density, were analyzed using an Instron 5848 with a 10N load cell. Elongation was assessed by cross-head displacement. Strips of 10 mm (length)×1 (width) mm, measured with a digital caliper were affixed to grips and pulled until failure at a strain rate of 1%/sec. For lung samples, parenchymal regions were analyzed, and the pleura was avoided. Hydration of samples with PBS was maintained before and during the mechanical conditioning. Using sample dimensions, engineering stress and strain were calculated using the following equations:

$$σ = \frac{F}{{A}_{0}} and ɛ = \frac{{l}_{f} - {l}_{o}}{{l}_{o}}$$

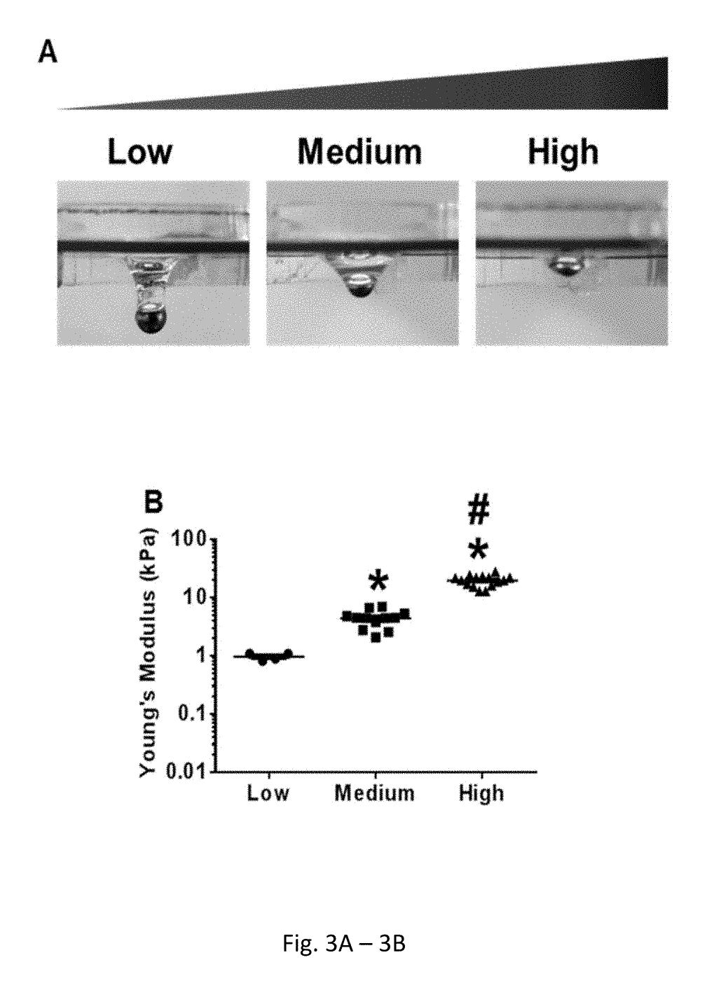

σ=stress, F=Force, A₀=initial area, ε=strain, l_{f}=final length, l₀=initial length. The Young's Modulus for the tissue was determined by dividing the engineering stress σ, by the engineering strain ε at 20% strain, within the normal range of lung inflation. Mechanical properties of the polyacrylamide hydrogels were also analyzed using a modified model of the Hertz equation using a custom made apparatus (FIG. 3) (Lauridsen et al., Technology, 2014, 2:133).

Myofibroblast Differentiation

For short term (7 day) differentiation studies, PC (passage 3-7) were seeded on healthy or IPF lung tissue slices, polyacrylamide hydrogels, or 25 mm glass coverslips (VWR) at 100×10³ cells per sample and activated with control media (non-activated), or 1 ng/mL TGF-β1 (PeproTech, Rocky Hill, N.J., USA) for 7 days (18, 19). For bleomycin activation, the cells were activated with 1 μg/mL bleomycin sulfate (Cayman Chemical, Ann Arbor, Mich.) for 1 day and the media was replaced with non-activated media for the remaining 6 days (Sava et al., Microcirculation, 2015, 22:54-67; Sterling et al., J Bio Chem, 1983, 258:14438-14444). TGF-β1 and bleomycin concentrations were based on reports consistent with activation of fibroblasts (Sava et al., Microcirculation, 2015, 22:54-67; Maroni and Davis, Bio Reprod, 2012, 87:127). Treatment with CCG-1423 (Sigma) was performed at day 0 for the full 7 days, and labeled as early treatment, or at day 7 and performed for 7 days, and labeled as late treatment. For long-term (28 day) differentiation studies, PC were cultured on 25 mm glass coverslips (VWR) or normal lung sections and the cell media was supplemented with 100 μM ascorbic acid (Spectrum Chemicals, New Brunswick, N.J., USA) to induce matrix deposition. Activation was performed with control media (non-activated), or 1 ng/mL TGF-β1. The media was replaced 3 times a week for 4 consecutive weeks (28 days) in order to mimic the chronic conditions present during fibrosis (Moeller et al., Int J Biochem Cell Biol, 2008, 40:362-382; Sava et al., Microcirculation, 2015, 22:54-67). Treatment with nintedanib (Santa Cruz) was performed at day 21 for the remaining 7 days, and labeled as early treatment, or at day 28 and performed for 2 days, and labeled as late treatment. Concentrations of CCG-1423 (1.0 nM), and nintedanib (1.0 nM) are based on therapeutic dose in studies performed on myofibroblasts (Evelyn et al., Mol Cancer Ther, 2007, 6:2249-2260, Hostettler et al., Resp Res, 2014, 15:157; Conte et al., Eur J Pharm Sci, 2014, 58:13-19; Shi et al., PLoS One, 2011, 6:e28134).

Pericyte pro-fibrotic activation/transition/phenotype was evaluated by positive staining for α-smooth muscle actin (SMA) using anti-α-SMA (1A4, Santa Cruz) and anti-vimentin (V630, Sigma). For matrix composition analysis, the coverslips were stained with anti-collagen I (ab233446, Abcam, Cambridge, Mass., USA), anti-laminin (Clone ab11575, Abcam), anti-collagen IV (Clone C1926, Sigma), or anti-fibronectin (Clone F3648, Sigma) antibodies.

Statistical Analysis

Data are expressed as mean ±standard error and were analyzed for significance by two-tailed Student t-test or one-way ANOVA with Tukey Post-Hoc test, with significance defined at p<0.05, indicated by *, or #.

The results are now described.

### Results

### Myofibroblasts Express Pericyte Markers in the IPF Lung

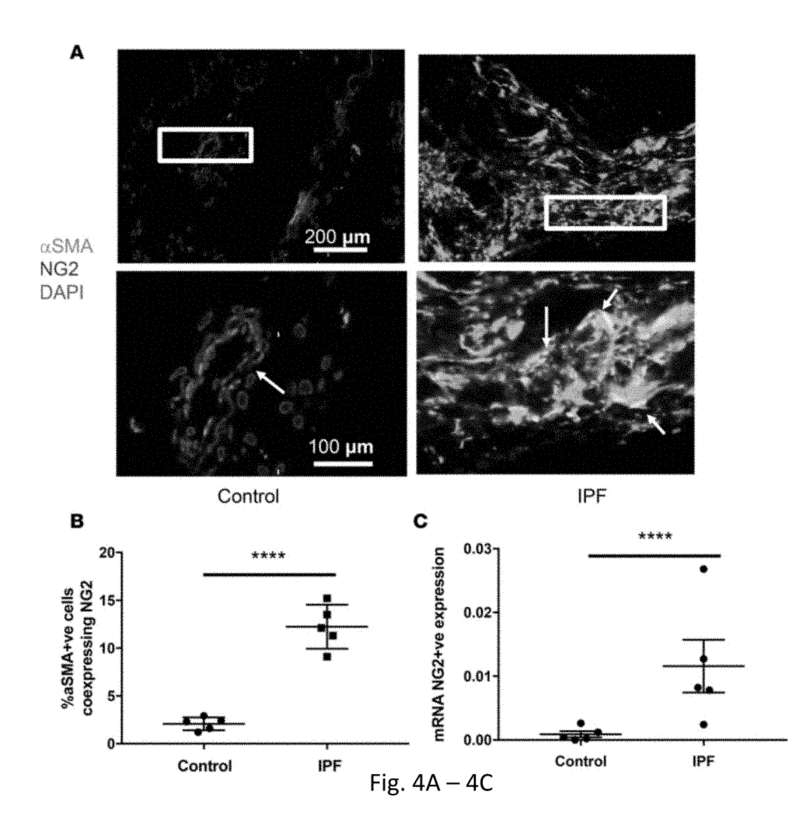

Myofibroblasts, characterized by their enhanced expression of α-SMA and ability to excessively remodel the extracellular matrix (ECM), are critical to the formation of fibrotic foci where fibrotic responses are initiated, and which are a relevant morphologic marker of idiopathic pulmonary fibrosis (IPF) (Chilosi et al., Resp Res, 2006, 7:95). While recent in vivo studies demonstrated that PC contribute 37% to 68% of myofibroblast population in bleomycin induced fibrosis, no direct evidence links PC to myofibroblasts in human IPF (Hung et al., Am J Respir Crit Care Med, 2013, 188:820-830; Kramann et al., Cell stem cell, 2015, 16:51-66). To begin to evaluate this question, lung tissues obtained from autopsies of patients who died of respiratory failure due to IPF, or died due to natural causes with histologically normal appearing lungs, were stained for myofibroblast marker α-smooth muscle actin (SMA). In control lungs, α-SMA was localized to the small and large blood vessels, indicating the presence of smooth muscle cells (FIG. 4A). The IPF lung contained similar regions, but also contained the presence of fibrotic foci, identified by the disruption of the normal lung architecture and circumferential orientation of α-SMA positive myofibroblasts (FIG. 4A). Co-staining of α-SMA+ myofibroblasts in the fibrotic foci with PC markers neural/glial antigen (NG)2 and CD90 revealed that a sub-population of myofibroblasts expressed the PC markers (FIGS. 4B-C). Notably, 21.6±2.5% percent of α-SMA+ myofibroblasts express NG2 and 17.1±2.1% percent of α-SMA+ myofibroblasts express CD90, as evaluated from representative images of fibrotic foci. Immunofluorescence staining of IPF lung supports in vivo findings and provides direct evidence that a sub-population of myofibroblasts in human IPF are of PC lineage.

### IPF Lung Sections Induced PC to Myofibroblast Transition

IPF manifests with the formation of mechanically stiff extracellular matrix, as previously shown through analysis of individual fiber mechanical properties (Booth et al., Am J Respir Crit Care Med. 2012, 186:866-876). Here, the composition and the bulk mechanical properties of the fibrotic lung were evaluated on samples obtained from autopsies performed within six hours of death through Yale's Rapid Autopsy Program. When comparing decellularized tissues prepared from lower lung of control patients or patients with IPF, Mason's Trichrome staining demonstrated that the ECM density of IPF lung is increased compared to the control lung (FIG. 4D). The increase in overall ECM content was largely due to an increase in highly fibrillar, cross-linked collagen I as shown by picrosirius red staining (FIG. 4D). The increase in ECM density led to a 11.6-fold increase in mechanical stiffness of the decellularized IPF lung (24.4±7.0 kPa) when compared to the control lung (2.1±0.5 kPa), as determined through tensile testing using an Instron 5848 system at 20% strain (FIGS. 4E-F).

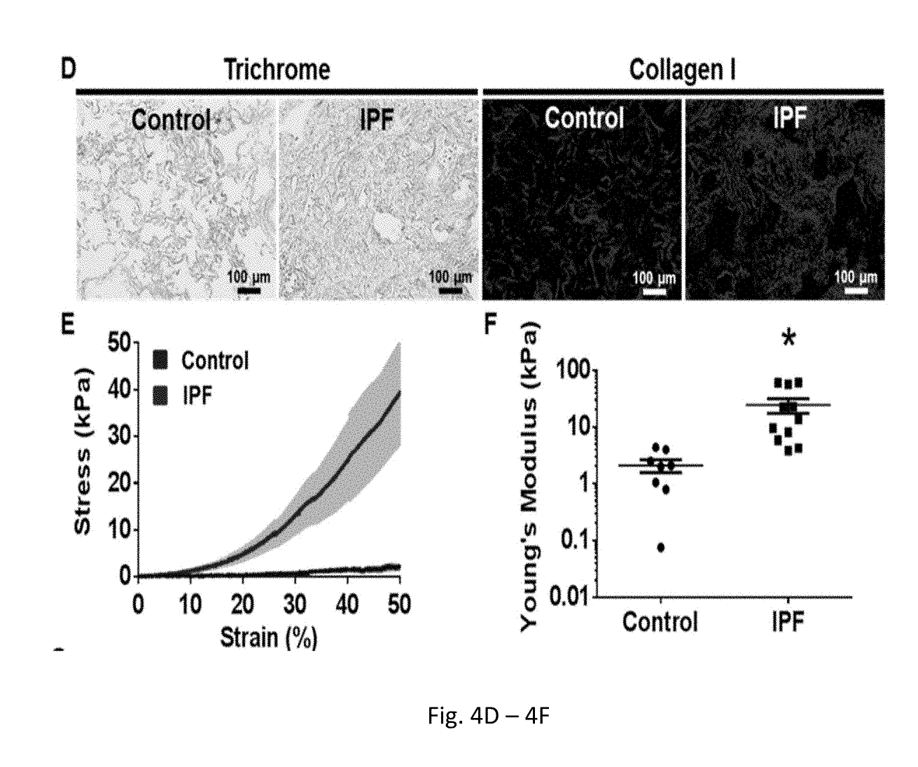

Previous studies have shown that decellularized tissue influence α-SMA tissue in cultured fibroblasts through mechanisms involving both biochemical and biophysical interactions (Booth et al., Am J Respir Crit Care Med. 2012, 186:866-876). In order to evaluate whether these properties might influence α-SMA expression by PC, several experiments were performed. First, the effect of a comparative difference in the composition and mechanical properties of control versus IPF lung on differentiation of PC into myofibroblasts was evaluated. Human microvascular PC were isolated from placental microvascular segments by explant outgrowth and screened by flow cytometry to confirm expression of NG2, CD90, CD146, PDGFR-β and α-SMA. Importantly, freshly isolated PC did not express smooth muscle cell markers (SMMHC-smooth muscle myosin heavy chain), endothelial cell markers (CD31, CD34), or leukocyte markers (CD45) (FIG. 1). Because human lung PC isolates are difficult to obtain in sufficient numbers for experimentation and, when isolated, are collected from tissue resection of lung cancer patients, an alternate and inexhaustible source of PC: namely, the human placenta was utilized. NG2/CD90/PDGFR-β/CD146-expressing human placental microvascular PC were cultured and then seeded onto decellularized IPF or control lungs and cultured for 7 days. Similar to studies of lung-derived fibroblasts, IHC-based evaluation of α-SMA revealed that, relative to cells grown on control samples, PC grown on decellularized IPF lungs increased expression of α-SMA (FIG. 4G), a finding that was confirmed by Western blotting (P=0.006, FIG. 4H). These data show that human PC respond to factors present in the fibrotic lung microenvironment to engender a population of α-SMA+ cells. A secondary myofibroblast marker, vimentin, was also examined, though no difference in expression was observed (FIG. 5). These data show that human pericytes cultured in the IPF ECM adopt a phenotype consistent with α-SMA expressing myofibroblasts.

### Progressive Matrix Stiffening Induces PC Activation in Normal Human Lung.

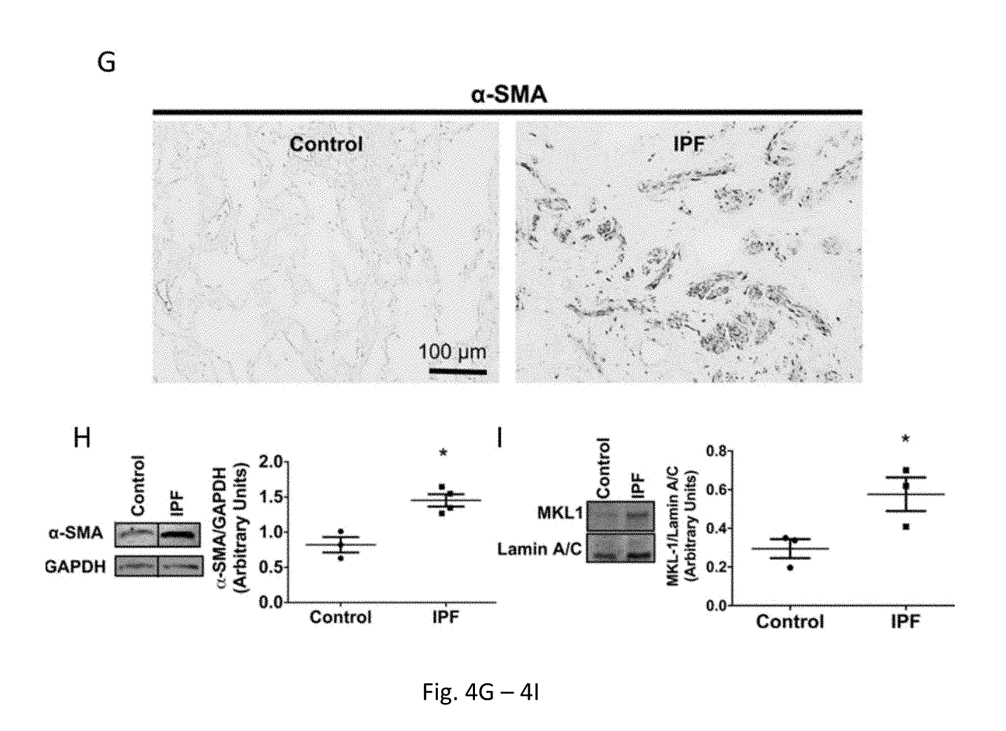

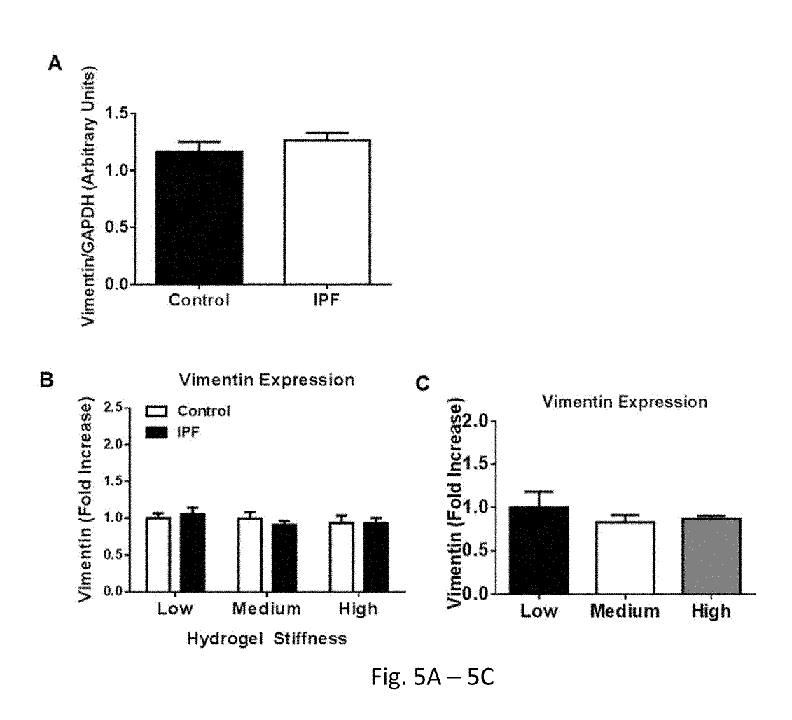

To determine whether these observations resulted from biophysical or biochemical interactions with the fibrotic matrix. Western blot was conducted to quantify nuclear translocation of megakaryoblastic leukemia 1 (MKL-1), also known as myocardin-related transcription factor A (MRTF-A). MKL-1 is a key transcription factor implicated in cellular responses to local stiffness. Nuclear translocation of MKL-1 was increased by 1.95-fold in PC cultured in IPF lung versus healthy lung (P=0.048, FIG. 4I), suggestive of active mechanosensing. To further study this process, we developed a culture platform that could provide the unique opportunity to decouple ECM stiffness and composition in the ex vivo setting. Here, polyacrylamide hydrogels of low (1.8±0.5 kPa), medium (4.4±0.5 kPa), and high (23.7±2.3 kPa) stiffness were generated to approximate the healthy, transitioning, and fibrotic human lung, respectively (FIGS. 6A and 6B, FIG. 2 and FIG. 3). These gels were then functionalized through chemical conjugation of solubilized ECM from control or IPF lung (FIG. 2 and FIG. 3). When PC seeded onto these substrates were analyzed after 7 days of culture (FIG. 6C), substrate stiffness was found to be the dominant driver of myofibroblastic properties. Specifically, relative to cells grown on the low-stiffness hydrogels, PC cultured on intermediate- and high-stiffness hydrogels displayed significantly increased cell area (FIG. 6D), elongation (FIG. 6E), and expression of α-SMA (FIG. 6F) in a manner that was independent of the source of conjugated ECM (control or IPF). Neither collagen I nor EDA fibronectin, both of which have been reported to induce α-SMA in fibroblasts, were capable of overcoming the effect of reduced matrix elasticity in our model (FIG. 7). Interestingly, there was no significant difference in α-SMA expression between cells cultured on intermediate- and high-stiffness hydrogels (FIG. 6F and FIG. 7). In further support of a mechanosensitive mechanism, nuclear translocation of MKL1 increased when PC were cultured on substrates of increasing stiffness, including both medium- (P=0.046) and high-stiffness (P=0.008) hydrogels (FIGS. 6, G and H). Furthermore, treatment with the MKL-1 inhibitor CCG-1423 at day 0 (P<0.001) or day 7 (P=0.039) of PC culture significantly reduced the detectable quantities of α-SMA (FIGS. 6I and 6J). These data indicate that even a small increase in substrate stiffness is sufficient to promote myofibroblast-like characteristics in human PC via mechanotransductive signaling. Vimentin expression was not altered for any of the conditions (FIG. 5).

There were no significant differences in α-SMA expression between PC cultured on the control lung and the IPF lung hydrogels, regardless of substrate stiffness (low, medium, or high stiffness). While not wishing to be bound by any particular theory, it is believed that the composition of the matrix does not directly induce PC to myofibroblast transition but that increased matrix stiffness is a more powerful activator. Previous studies have demonstrated the role of fibronectin containing the extra domain A (EDA) in inducing fibroblast to myofibroblast transition in vitro and have demonstrated its importance in IPF in vivo (Bhattacharyya et al., Sci Transl Med, 2014, 6:232ra250; Kohan et al., FASEB, 2010, 24:4503-4512). However, these in vitro studies have used either soluble EDA-FN supplemented in culture media or have been performed on a non-physiologically stiff substrate (glass). To confirm the findings that ECM composition does not induce PC to myofibroblast transition, EDA-FN were isolated from pericyte conditioned media using gelatin affinity chromatography, confirmed through western blot analysis, and cultured PC for 7 days on either 0.1 mg/mL EDA-FN or 0.5 mg/mL EDA-FN conjugated to hydrogels of low, medium, and high stiffness. Another important ECM protein in fibrosis, collagen I, was also conjugated to hydrogels (FIG. 7). Overall, neither increased concentrations of EDA-FN or collagen I induced a significant increase in PC α-SMA expression at low, medium, or high stiffness, further supporting that PC to myofibroblast transition is largely driven by matrix stiffness and not composition.

### Matrix Stiffness Induced Myofibroblast Transition is a Result of MKL1 Signaling

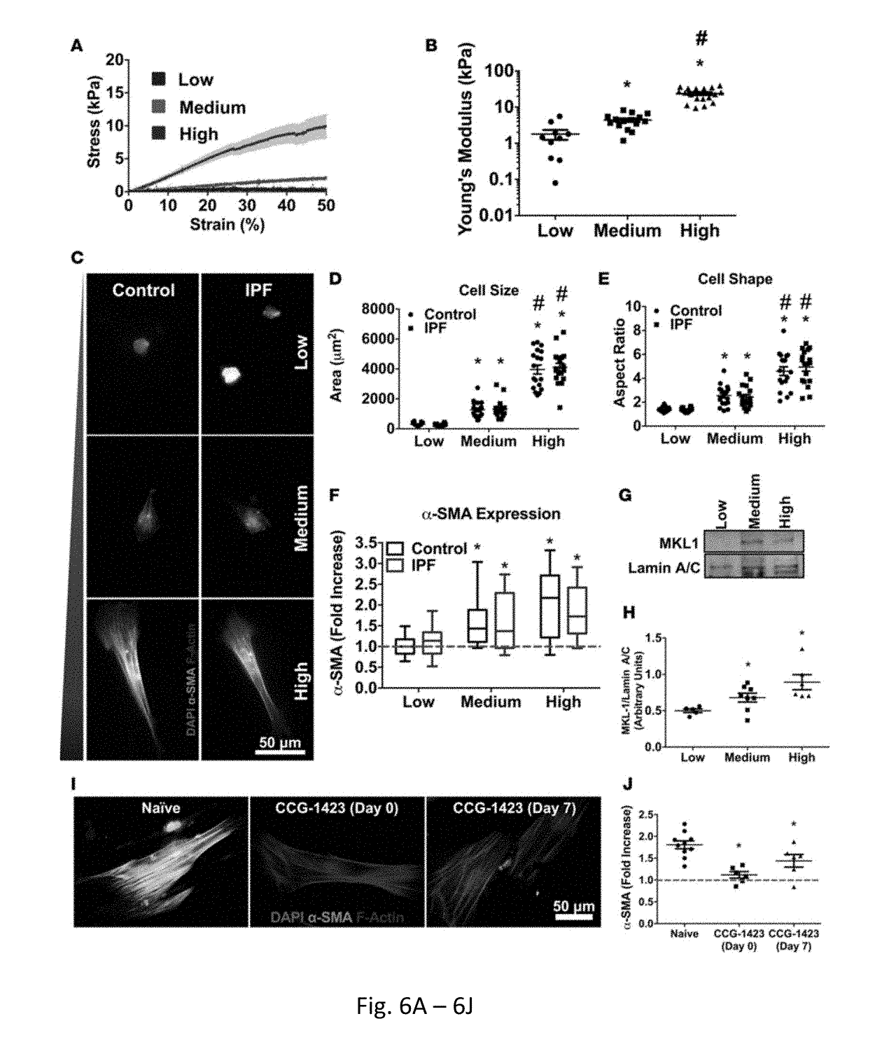

To more closely investigate the role of matrix stiffness on pro-fibrotic transition, without the complication of multiple proteins found in the complex entirety of the tissue, a minimal adhesive ECM preparation of fibronectin and collagen I (1:1 ratio) was used. PC were cultured on the varying stiffness hydrogels, fixed and stained for F-Actin, α-SMA, and vimentin expression. As was seen on the healthy lung hydrogels, increased mechanical stiffness of the substrate induced an increase in cell area and aspect ratio, inducing a morphological shift from a circular cell on the low stiffness substrate, to an elongated and extended cell on high stiffness substrate (FIGS. 8A-C). Additionally, increased elastic modulus of the substrate increased α-SMA expression, with the medium stiffness substrate demonstrating a 56.9±17.0% increase and the high stiffness substrate demonstrating a 72.1±8.4% increase in α-SMA expression compared to the low stiffness substrate (FIGS. 8D-E). However, the increase between medium and high stiffness hydrogels was not significant. These findings were supported by western blot analysis, while vimentin expression was not altered (FIG. 5).

While the mechanism underlying PC mechanosensing have not been evaluated, nuclear translocation of megakaryoblastic leukemia (MKL1) is responsible for the stiffness induced increase in α-SMA in fibroblasts, and has been associated with fibroblast-to-myofibroblast differentiation. MKL1 operates through outside-in signaling pathways regulated, in part by integrin mediated mechanotransduction and promotes activation of the RhoA (Ras homolog gene family member A)/ROCK (Rho-associated protein kinase) pathway (Huang et al., Am J Respir Cell Mol Biol, 2012, 47:340-348). In order to evaluate the contribution of MKL1, PC nuclear protein was extracted and nuclear translocation of MKL1 was detected by western blot analysis to infer its responsibility for the stiffness induced increase in α-SMA expression. PC cultured on the human IPF lung section exhibited an increase in MKL1 nuclear translocation by 95.5±29.5% compared to PC cultured on control lung scaffolds (FIGS. 8F-G). Further, nuclear translocation of MKL1 increased in response to increased substrate stiffness as PC cultured on the high stiffness hydrogel demonstrated a 78.4±20.7% increase in MKL1 translocation compared to PC cultured on the low stiffness hydrogel (FIGS. 8H-I).

Since increased mechanical stiffness is largely responsible for the increase in PC to myofibroblast transition and drives this process in an MKL1 dependent manner, the effectiveness of MKL1 targeted therapeutic strategies to prevent or reverse stiffness mediated fibrotic progression was investigated. CCG-1423 has been demonstrated to inhibit the interaction of MKL1 with the serum response factor (SRF) and has been effective in preventing fibrosis in in vivo mouse models (Evelyn et al., Mol Cancer Ther, 2007, 6:2249-2260; Ho et al., Nat Rev Rheumatol, 2014, 10:390-402). PC cultured on the high stiffness hydrogel were treated with CCG-1423 for 7 days at an early time-point (day 0) or following stiffness induced progression at a late time point (day 7) for an additional 7 days, and α-SMA expression was analyzed using flow cytometry. F-Actin staining demonstrated that CCG-1423 treatment was specific in blocking MKL1 action in the nucleus and did not alter cell F-Actin architecture (FIG. 8J). Treatment with CCG-1423 at an early time-point of PC culture significantly decreased the expression of α-SMA by 38.2±4.2%, maintaining levels of α-SMA expression of PC cultured on low stiffness hydrogel (FIG. 8K). Treatment with CCG-1423 at a late time point significantly decreased the expression of α-SMA by 20.4±8.0%. These data identify that MKL1 involvement in the early stages of PC to myofibroblast transition, and suggest that it is possible for myofibroblast differentiation to be partially reversed by inhibiting the mechanosensing ability of the cells.

### Bleomycin Activation Induces PC to Myofibroblast Differentiation

Matrix remodeling and altered mechanical stiffness do not occur in isolation, and often correlate with increased TGF-β1 production in human disease and animal models of lung fibrosis. TGF-β1 is an inducer of myofibroblast transition in fibroblasts and thereby a potent activator of matrix deposition that leads to matrix stiffening (Pu et al., Yale J Biol Med. 2013, 86:537-554). Alternatively, bleomycin is an experimental reagent used to induce TGF-β1-replicative lung fibrosis in mice (Wynn and Ramalingam, Nat med. 2012, 18:1028-1040; Wynn, J Exp Med. 2011, 208:1339-1350; Harrison and Lazo, J Pharmacol Exp Ther, 1987, 243:1185-1194; Phan et al., J Clin Invest, 1985, 76:241-247). While human microvascular PC are responsive to both TGF-β1 and bleomycin, the effects of these interventions on matrix deposition vary, suggesting that the PC offer an ideal cell type to study activator specific fibrotic progression (Sava et al., Microcirculation, 2015, 22:54-67).

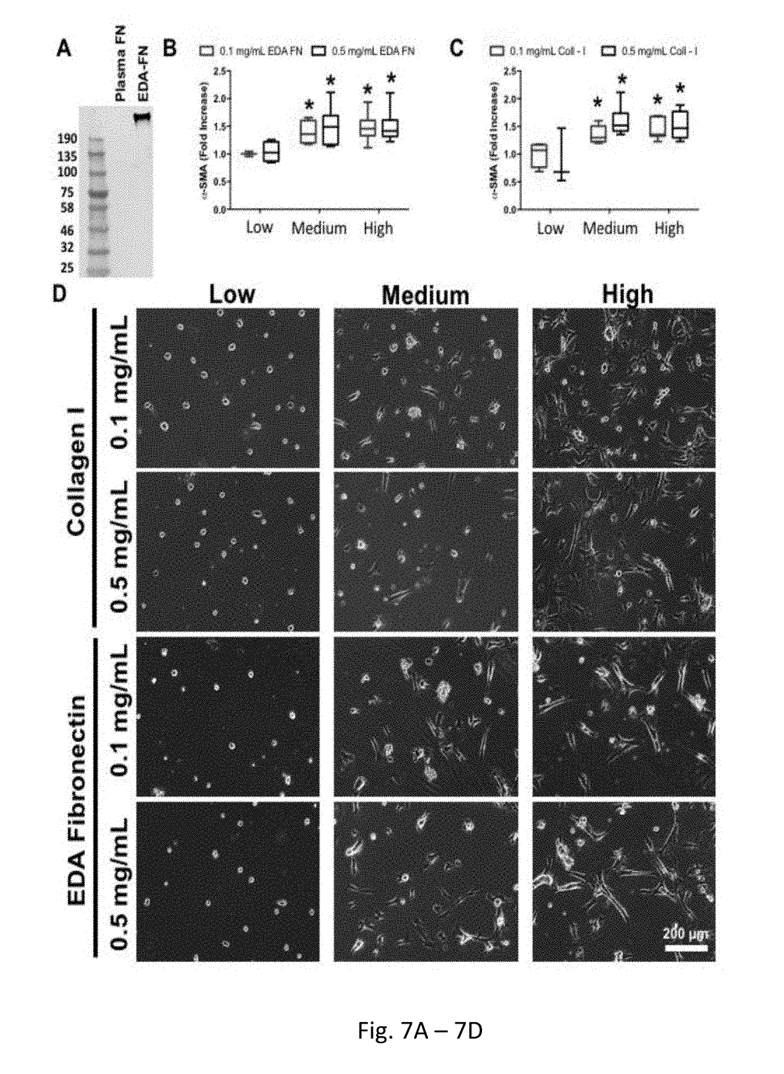

Here, the combinatorial effect of altered mechanical stiffness and TGF-β1 or bleomycin mediated activation are investigated. PC were cultured on polyacrylamide hydrogels of low, medium, or high stiffness conjugated to a 1:1 ratio of fibronectin and collagen I. The PC were activated with either TGF-β1 for 7 continuous days, or bleomycin for 1 day, followed by 6 days of no activation as previously described (Sava et al., Microcirculation, 2015, 22:54-67). As seen during the non-activated condition, increased stiffness significantly increased the cell area and aspect ratio of both TGF-β1 and bleomycin activated PC, with bleomycin activation resulting in cells with the largest area and elongation on the medium and the high stiffness substrates (FIGS. 9A-C). Following TGF-β1 activation, PC had a gradual increase in α-SMA expression with increasing stiffness (FIG. 9D). α-SMA expression of TGF-β1 activated PC on the medium and high stiffness hydrogels was significantly increased compared to the PC on the low stiffness hydrogel, while vimentin expression was not altered (FIGS. 9E-F). However, the TGF-β1-mediated activation did not induce a significant increase in α-SMA or vimentin expression compared to non-activated PC cultured on substrates of the same stiffness. In contrast, bleomycin activation significantly increased α-SMA and vimentin expression when compared to non-activated PC cultured on low, medium, and high stiffness substrates (FIGS. 9D-F). The effect of matrix stiffness was negated by bleomycin-mediated activation, as α-SMA and vimentin expression were not significantly increased across stiffness. In these short-term studies, it was discovered that TGF-β1 activation did not induce PC to myofibroblast transition. However, bleomycin activation was a potent inducer of PC to myofibroblast transition and overrode the stiffness mediated effect.

### Exposure to Exogenous TGF-β1 Does Not Influence Accumulation of α-SMA⁺ PC.

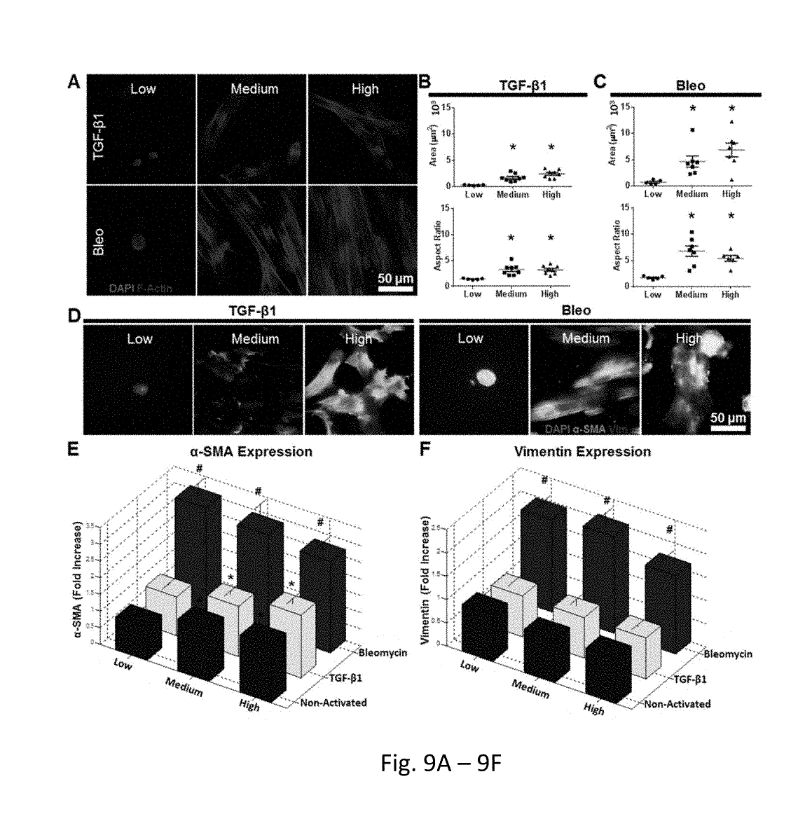

TGF-β1 acts as a central mediator of fibrosis that regulates the fibroblast-to-myofibroblast transition, though its role in mechanotransductive processes has been incompletely explored. To determine whether exposure to TGF-β1 affects expression of α-SMA, freshly isolated PC were grown on soft or stiff hydrogels for up to 7 days in the presence or absence of TGF-β1. Interestingly, activation with TGF-β1 failed to affect the proportion of α-SMA-expressing PC cultured on hydrogels of any stiffness (FIG. 11A and FIG. 11B). These data indicate that, unlike fibroblasts (Hu et al., (2003) Am J Respir Cell Mol Biol, 29(3 Pt 1):397-404), short-term exposure to TGF-β1 is insufficient to oppose the effects of substrate stiffness, further supporting a central role for mechanosensing in the initial accumulation of α-SMA-expressing PC.

### TGF-β1 Activation Induces PC Matrix Deposition.

Current paradigms of lung fibrosis propose that the TGF-β1-rich milieu occurring in the later stages of fibrosis favors the accumulation and activation of matrix-producing myofibroblasts (Wynn and Ramalingam, (2012) Nat Med. 2012;18(7):1028-1040). The bioinert and hydrophilic nature of our hydrogel does not allow for examination of PC matrix deposition. Therefore, a system was developed in which PC grown on glass allows accumulation and evaluation of deposited ECM. The PC were exposed to TGF-β1 for up to 28 days as a model of the chronic exposure to fibrogenic stimuli that would be encountered in the fibrotic human lung (FIG. 11C). Without being bound by a particular theory, it was hypothesized that an alternate effect of TGF-β1 might involve a potentially novel and previously unrecognized autocrine contribution to ECM deposition by microvascular cells. This prediction proved accurate, as—following long-term activation—PC responded to TGF-β1 by producing circumferential nodes of ECM consisting of a central nucleus high in collagen I and collagen IV, surrounded by a radiating matrix that was rich in fibronectin (FIG. 11C).) Node formation was first detected after 7 days of TGF-β1 exposure, with a 2.2-fold (P<0.001) and 1.8-fold (P<0.01) increase in the formation of lesions at days 14 and 28, respectively, compared with the nonactivated control (FIG. 11C through FIG. 11E). The number of nodes following TGF-β1 activation peaked at day 14 and decreased by day 28, likely due to a ripening effect wherein individual foci coalesce to become larger, singular foci (FIG. 11E). Immunofluorescence evaluation revealed that cells within the nodes expressed high levels of α-SMA and vimentin compared with the surrounding cells (FIG. 11F). Interestingly, in this model, TGF-β1-activated PC exhibited a significant increase in both α-SMA (P=0.011) and vimentin (P<0.0001) expression compared with the nonactivated control (FIG. 11F through FIG. 11I). These data indicate that PC respond to TGF-β1 by adopting functional characteristics of myofibroblasts, including the ability to produce a collagen and fibronectin-rich ECM, which in turn facilitates the increased expression of α-SMA and vimentin by these highly plastic cells.

### TGF-β1-Induced Stimulation of PC Matrix Production is Opposed by Treatment With Nintedanib.

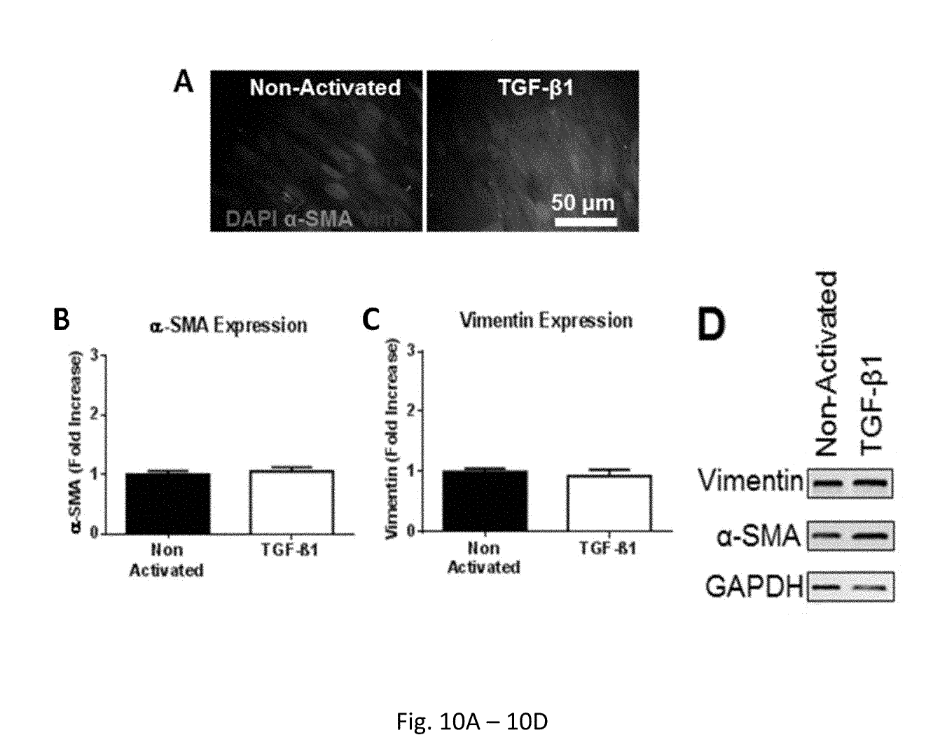

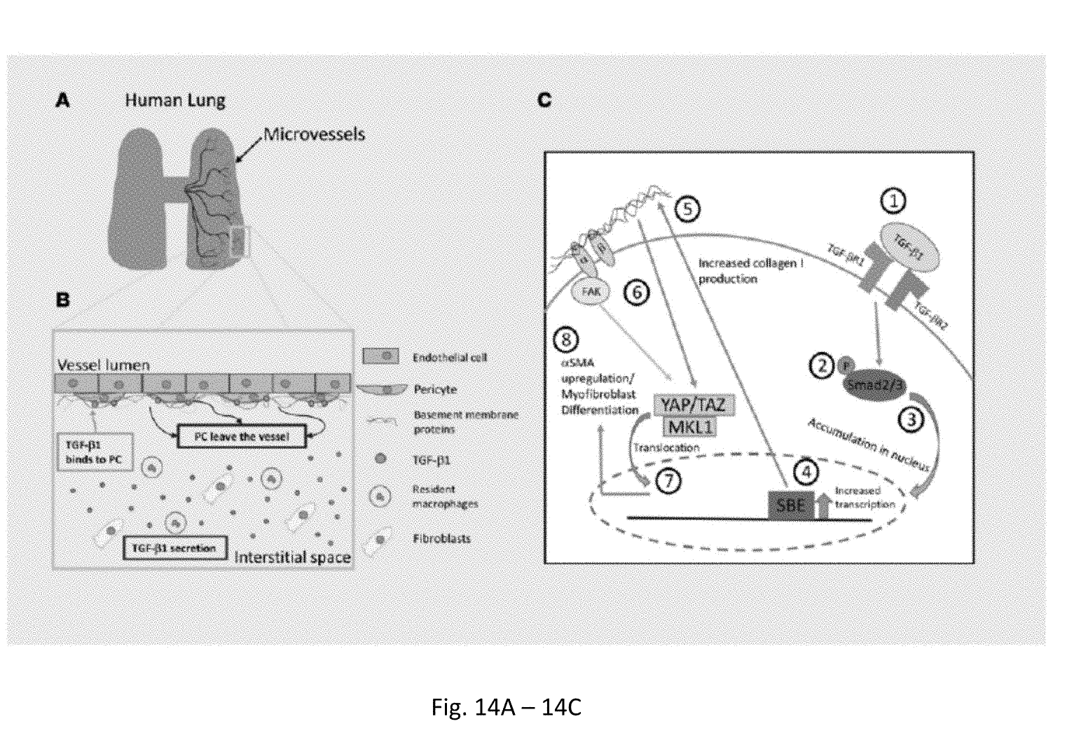

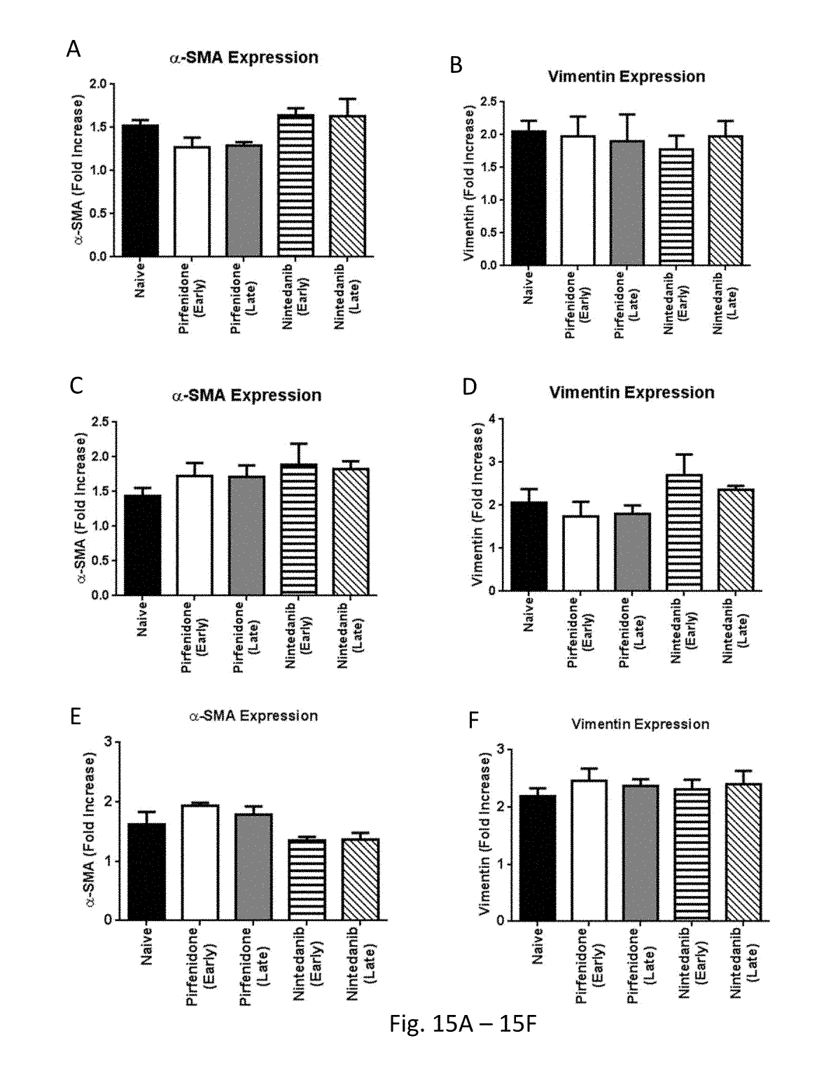

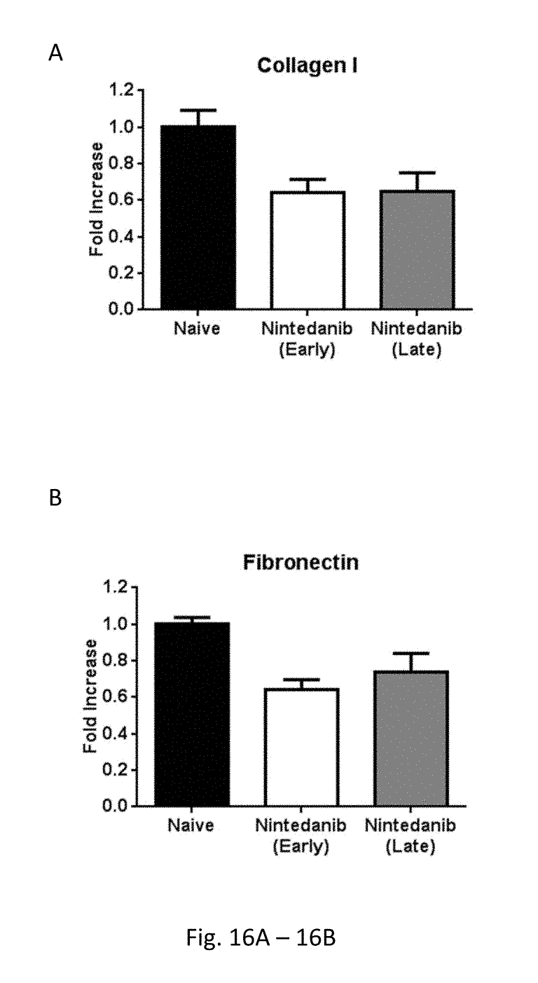

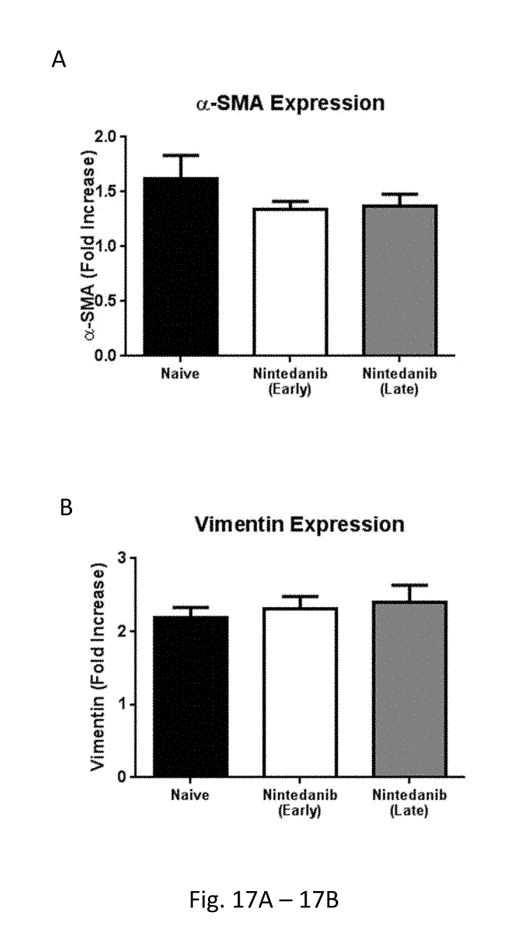

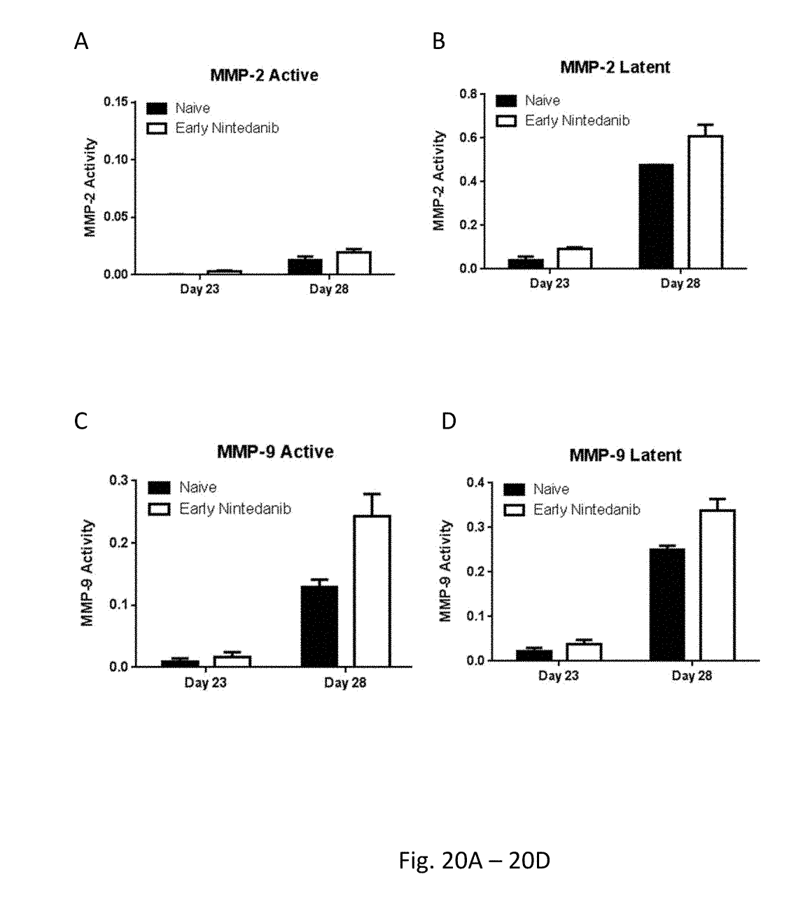

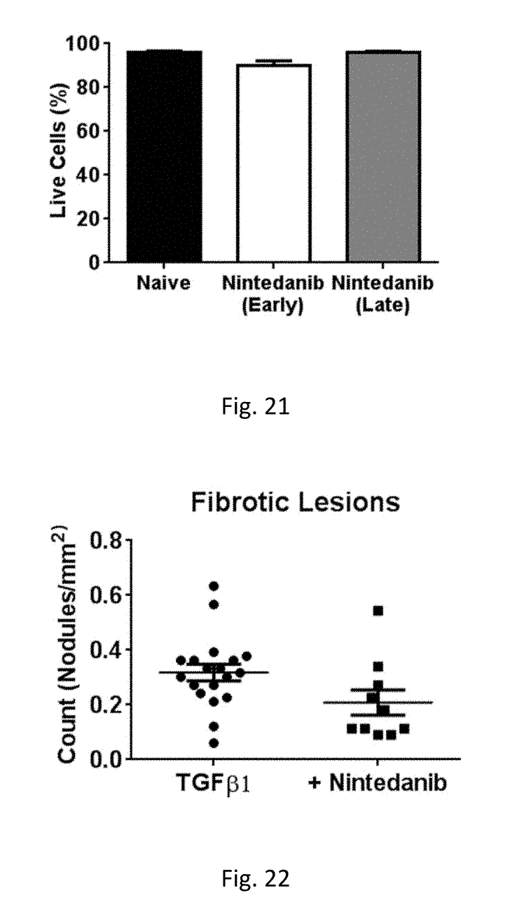

Currently available IPF therapeutics, including the small molecule pyridone derivative pirfenidone and the tyrosine-kinase inhibitor nintedanib, can delay but not prevent or reverse the progression of fibrosis (Myllarniemi and Kaarteenaho (2015), Eur Clin Respir J, 2). Nintedanib is known to oppose TGF-β1-induced myofibroblast differentiation and matrix deposition of cultured lung fibroblasts in vitro (Rangarajan et al., (2016) Am J Respir Cell Mol Biol, 54(1):51-59), but its effect on these endpoints in PC has not been defined. In order to address this issue, α-SMA⁻, vimentin⁻, and matrix-producing PC were generated via long-term exposure to TGF-β1 as described above, and they were subsequently treated with TGF-β1 with or without nintedanib for an additional 7 days (FIG. 12 and FIG. 16 through FIG. 19). Importantly, nintedanib substantially reduced detection of ECM components (FIG. 12A through FIG. 12E) in a manner that was independent of effects on α-SMA or vimentin (FIG. 12F through FIG. 12H). Specifically, nintedanib treatment reduced collagen I (P=0.01) and fibronectin (P=0.002) fibrotic node formation compared with the increase observed following TGF-β1 activation of PC (P=0.049, FIG. 12A through FIG. 12E, FIG. 21, FIG. 22) without affecting the expression of α-SMA or vimentin (FIG. 12F and FIG. 12G). These data demonstrate a potentially novel and previously unrecognized mechanism of action of nintedanib that involves the regulation of ECM production by PC-derived myofibroblasts.

### TGF-β1-Induced Lung Remodeling Properties of α-SMA⁺ PC are Reduced by Nintedanib.

The studies described above present compelling evidence of the ability of PC to modify and respond to their local microenvironment. However, because these studies were performed on glass substrates, their relevance to the human lung remains uncertain. The ability of PC to cause pathologic remodeling and ECM accumulation was evaluated in a more realistic culture system: namely, the decellularized human lung. PC were seeded on scaffolds prepared from decellularized healthy lungs, activated with TGF-β1 for 21 days, and further stimulated cells with TGF-β1 in the presence or absence of nintedanib. Masson's trichrome and Picosirius red staining revealed that treatment of PC with 28 days of TGF-β1 increased both ECM density and collagen I crosslinking (FIG. 12I and FIG. 12J). These findings were accompanied by an 8.6-fold increase in the elastic modulus of the scaffold to levels similar to those of IPF lungs (P=0.001, FIG. 12J). Importantly, in this model, treatment with nintedanib reduced the number of PC expressing α-SMA by 90.1% (P<0.0001, FIG. 12K and FIG. 12L) in a manner that was accompanied by a reduction in both matrix density and collagen I content (FIG. 12I). Treatment with nintedanib also caused reversion of the elastic modulus to baseline levels (P=0.44 compared with control lung; P=0.001 compared with TGF-β1 without nintedanib-treated lung) (FIG. 12J). These data show that TGF-β1-stimulated PC can remodel otherwise normal lung ECM and that these changes are opposed by treatment with nintedanib.

### Mechanism of Nintedanib Therapeutic Function in PC-Induced Lung Fibrosis.

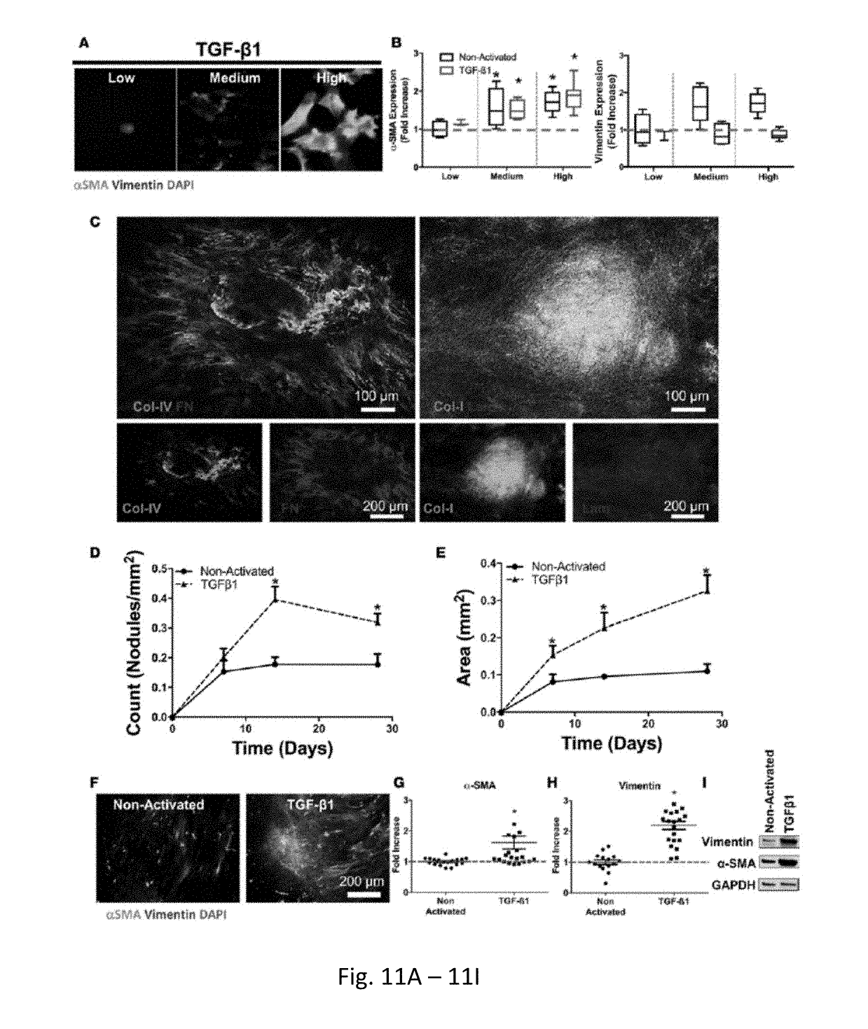

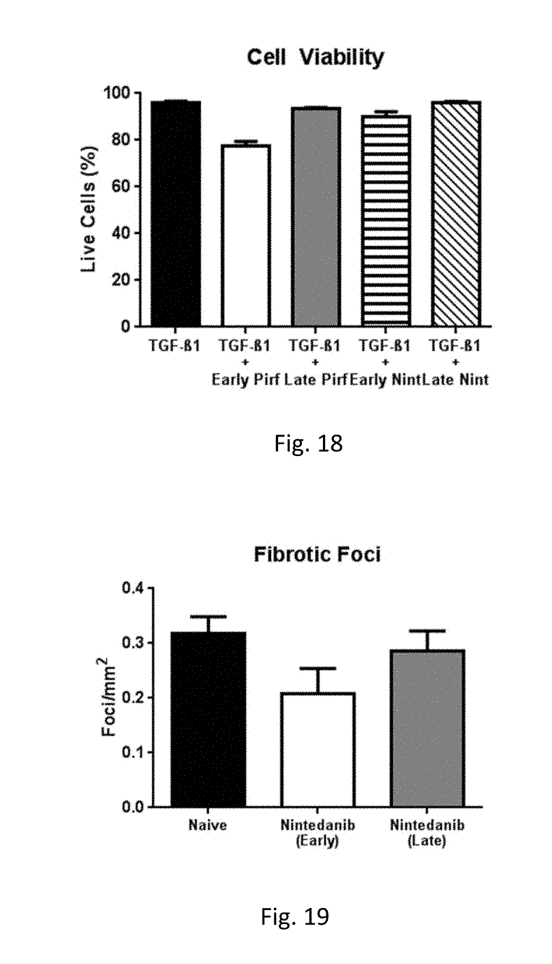

The mechanism of nintedanib efficacy is incompletely defined and, thus far, has ignored any effects on PC. Because treatment with nintedanib was able to reduce the stiffness of fibrotic lung matrices, we reasoned that its mechanism of action in this context must involve degenerative remodeling of the ECM, perhaps through the secretion and activation of matrix metalloproteinases (MMPs). Thus, supernatants recovered from TGF-β1-activated PC were subject to ELISA-based detection of MMP8, -9, and -13, which revealed substantial increases in all 3 mediators following nintedanib treatment (P<0.05, P<0.01, and P<0.001, respectively, compared with treatment with TGF-β1 alone; FIG. 13A through FIG. 13D). Nintedanib treatment did not increase expression of MMP10 (FIG. 13E). Finally, generation of active collagenases MMP2 and MMP9 was also enhanced when TGF-β1-activated PC were treated with nintedanib (P<0.05 and P<0.01, respectively; (FIG. 13F, FIG. 13G and FIG. 20), providing evidence for the matrix remodeling and increased lung tissue elasticity previously observed. Together, these data demonstrate the unexpected ability of PC to respond to and modify their microenvironment, demonstrating both the regulated production of ECM and the generation of matrix-degrading MMPs in response to exogenous stimuli. They also extend the mode of action of IPF therapies to include the regulation of PC-mediated matrix remodeling events via a mechanism involving production and activation of MMPs.

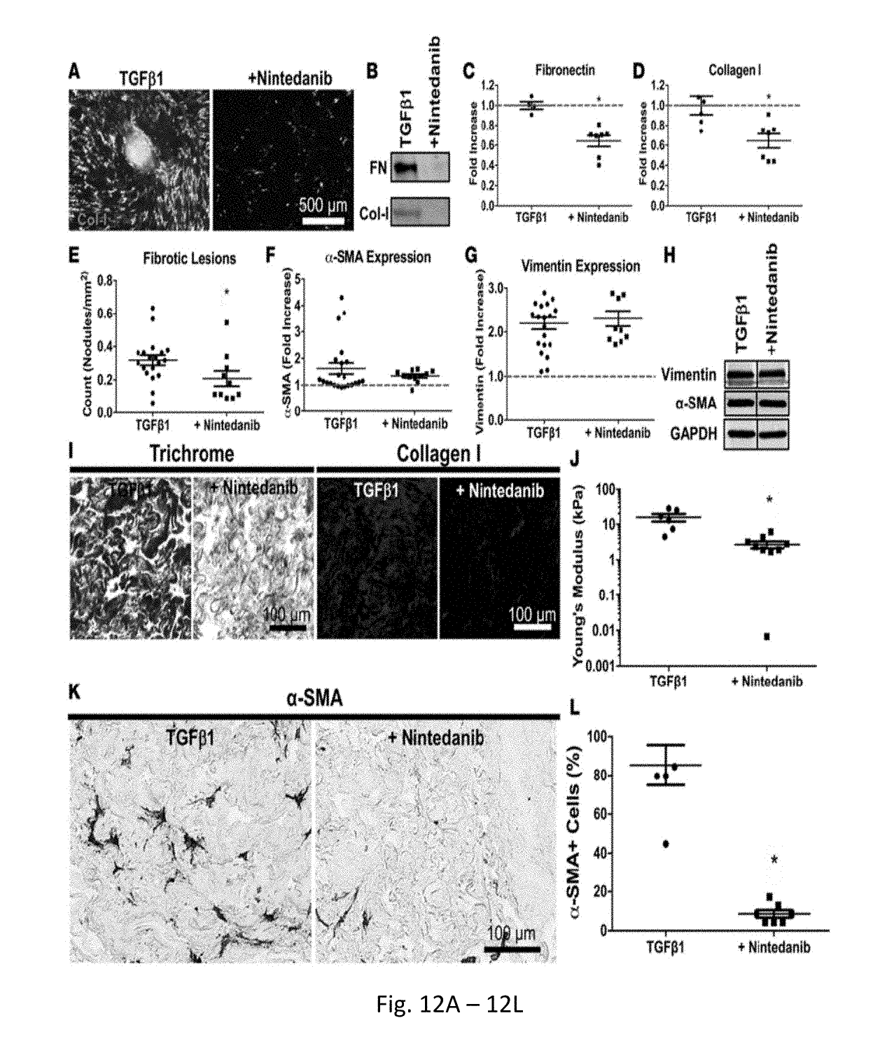

These data provide important new insight into the role of PC in the development and perpetuation of fibrosis and remodeling of the human lung. A subpopulation of α-SMA-expressing IPF lung myofibroblasts express PC markers, thereby showing, for the first time to our knowledge, a contribution of PC to fibroproliferative lung disease in humans. In an ex vivo corollary of these findings, we used an experimental platform based on primary human cells and tissues to decouple the biochemical and biomechanical regulation of PC phenotype. The accumulation of α-SMA-expressing PC was driven by contact with the diseased ECM in an MKL1-dependent mechanotransductive process that is largely independent of ECM composition. Moreover, this study aims to highlight the importance of mechanosensing pathways for the accumulation of α-SMA⁺ PC in the fibrotic foci. However, these pathways, while well studied in fibroblasts, remain to be confirmed in PC. For example, focal adhesions and focal adhesion kinases (FAK) play an integral part of α-SMA expression in fibroblast-to-myofibroblast transdifferentiation (Pu et al., (2013) Yale J Biol Med, 86(4):537-554) as a means of signal transduction from clustered integrin-ligand binding interactions between cells and the fibrotic ECM. Additionally, immunostaining of fibroblasts grown on fibronectin-coated gels with varying stiffness revealed an increase in nuclear translocation of yes-associated protein (YAP) and transcriptional coactivator with PDZ-binding motif (TAZ) in fibroblasts seeded on stiffer substrates (Szeto et al., (2016) J Am Soc Nephrol. 27(10):3117-3128). A YAP/TAZ-dependent mechanism in PC following TGF-β1 activation has yet to be determined (FIG. 14). While short-term treatment of PC with TGF-β1 does not alter α-SMA expression, longer-term TGF-β1 exposure expands the α-SMA⁺, vimentin⁺ population capable of producing excess ECM. These cells also remodeled decellularized normal lung scaffolds through collagen and fibronectin deposition and enhancement of matrix stiffness. This effect is reversible through treatment with the tyrosine-kinase inhibitor nintedanib via production of MMPs. Altogether, these data support the contention that PC-derived myofibroblasts arise in response to altered tissue stiffness to contribute to lung remodeling and ECM accumulation in IPF.

Ex vivo analysis shows that local substrate stiffness regulates the early events in accumulation of α-SMA⁺ PC, as cells seeded into the decellularized IPF lung show increased engraftment and persistence of α-SMA⁺ cells compared with those seeded into control lung scaffolds. Interestingly, these events are regulated by cellular responses to local substrate stiffness and not solely as an effect of receptor-ligand interactions with specific biochemical components of the IPF ECM. Even small increases in the elastic modulus are sufficient to drive the α-SMA⁺ phenotype, suggesting that the severity of matrix stiffening seen in the end-stage IPF lung is not necessary to drive PC accumulation. Since stiffness, and not matrix composition or TGF-β1, drove the initial accumulation of α-SMA⁺ PC, these findings suggest that any condition that increases the elastic modulus—such as other forms of fibrotic lung disease, acute respiratory distress syndrome (ARDS), occupational lung disease, ventilator induced lung injury, and even normal aging—might be sufficient to expand PC in this manner.

The results of the experiments provided indicate that short-term exposure to TGF-β1 is insufficient to induce α-SMA in cultured PC. This characteristic differs markedly compared with what has been reported for resident lung fibroblasts, which are known to adopt α-SMA expression after as little as 48 hours of TGF-β1 exposure (Thannickal et al., (2003) J Biol Che. 278(14):12384-12389) suggesting that PC are a unique cell population that are functionally distinct from mature lung fibroblasts. In fact, it is after long-term exposure to TGF-β1 that PC adopt functional properties of myofibroblasts. These data suggest several competing hypotheses: that either all PC display low-level responsiveness to TGF-β1 that increases over time, that all PC display endogenous mechanisms to dampen TGF-β1 activation that are lost over time, or that only a small fraction of PC are able to respond to TGF-β1 and that they do so by producing a collagen- and fibronectin-rich ECM that drives additional accumulation of α-SMA expressing PC via mechanotransductive responses to such ECM. Without being bound by a particular theory, but in view of the finding that α-SMA⁺ PC expand in response to biomechanical influences (FIG. 14), the latter hypothesis is preferred, though dedicated studies aimed at evaluating TGF-β1 receptor expression and signaling pathways will be required to fully answer this question.

These studies also present a potentially novel mode of action for the antifibrotic agent nintedanib. Most studies of nintedanib have considered its effect on resident lung fibroblast, and we are the first to our knowledge to propose an additional effect on PC biology. While nintedanib has been reported to inhibit tumor growth by reducing PC coverage (Kutluk et al., (2013) Mol Cancer Ther. 12(6):992-1001), the results provided show a connection between this triple kinase inhibitor and PC in the setting of diffuse parenchymal lung disease. Treatment with nintedanib was sufficient to reduce the expression of α-SMA, to reduce the detection of cross-linked collagen, and to lessen the elastic modulus in long-term cultures of control lung scaffold that had been seeded with TGF-β1 stimulated PC. These data suggest that rather than acting directly on TGF-β1-induced myofibroblast transformation, as has been proposed for postembryonic lung fibroblasts (Kimura et al., (2016) Respir Res, 17:19), nintedanib's effects on PC might be exerted via inhibiting the amplification of fibrosis caused by the diseased ECM. This effect was partially due to MMPs, though other mechanisms cannot be ruled out. Further, the effect of combinatorial therapeutic approaches to trigger synergistic matrix remodeling and mechanosignaling was not evaluated in this study but may be impactful. The data suggest that PC are capable of degenerative tissue remodeling through MMP generation observed in both vascular smooth muscle cells and fibroblasts. These PC have the capacity to behave both in a vascular role and interstitial fibroblast-like role. Further investigation of this finding may lead to better understanding of cell-matrix interactions in a variety of clinical settings beyond IPF.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a tunable hydrogel, the hydrogel comprising a polymer component and an extracellular matrix (ECM) component, wherein the hydrogel is formed by the step of employing an amount of a crosslinking agent to achieve a Young's elasticity modulus of the substrate that ranges from 0.1 to 150 kPa.

2. The composition of claim 1, wherein the polymer comprises polyacrylamide, and the crosslinking agent comprises bis-acrylamide.

3. The composition of claim 1, wherein the ECM component is one or more of decellularized, solubilized and conjugated to the hydrogel.

4. The composition of claim 3, wherein the ECM component is chemically conjugated to the hydrogel.

5. The composition of claim 1, wherein the ECM component is pericyte derived (PC-ECM).

6. The composition of claim 1, wherein Young's elasticity modulus is selected from about less than 1 kPa to about 40 kPa.

7. The composition of claim 1, wherein Young's elasticity modulus is selected to mimic a tissue.

8. The composition of claim 6, wherein a tissue is healthy lung and Young's elasticity modulus is selected to be about 2 kPa.

9. The composition of claim 6, wherein a tissue is a fibrotic lung and Young's elasticity modulus is selected to be greater than 20 kPa.

10. A method of generating a composition of claim 1, comprising selecting a ratio of polymer to crosslinking agent such that the resulting hydrogel comprises a Young's elasticity modulus selected to mimic a tissue; contacting the selected amount polymer with the selected amount of crosslinking agent in the presence of light and an aqueous solution to form a hydrogel; and contacting the hydrogel with decellularized, solubilized ECM.

11. The method of claim 10, wherein the method further comprises setting the elasticity of the substrate by selecting a concentration of cross-linking density within the matrix, such that Young's elasticity modulus is adjustable over several orders of magnitude, from extremely soft to stiff.

12. The method of claim 10, wherein the hydrogel comprises a hydrogel component and an ECM component, wherein the ECM is decellularized, solubilized and conjugated to the hydrogel.

13. The method of claim 10, wherein the ECM is PC-ECM.

14. A method for culturing an anchorage-dependent cell, comprising: providing a substrate having an elasticity defined by Young's elasticity modulus; introducing the anchorage-dependent cell onto said substrate; and culturing the anchorage-dependent cell.

15. The method of claim 14, wherein the substrate comprises a tunable matrix formed by the step of employing an amount of a crosslinker to achieve a finely tunable Young's elasticity modulus of the substrate that ranges from 0.1 to 150 kPa.

16. The method of claim 15, wherein the substrate comprises polyacrylamide, and the crosslinker comprises bis-acrylamide.

17. The method of claim 14, wherein Young's elasticity modulus is selected from about less than 1 kPa to about 40 kPa.

18. The method of claim 14, wherein Young's elasticity modulus is selected to mimic a tissue.

19. The method of claim 18, wherein a tissue is selected from the group consisting of healthy lung and a fibrotic lung, wherein when tissue is healthy lung the Young's elasticity modulus is selected to be about 2 kPa, and wherein when tissue is a fibrotic lung the Young's elasticity modulus is selected to be greater than 20 kPa.

20. A kit comprising a composition of claim 1.
